(19) United States
(12) Patent Application Publication
(10) Pub. No.: US 2026/0286417 A1
(43) Pub. Date: Sep. 24, 2026

(54) COMPOSITION AND METHOD FOR BACTERIOPHAGE SUSCEPTIBILITY SCREENING

(71) Applicant: MCMASTER UNIVERSITY, West Hamilton (CA)

(72) Inventors: Zeinab HOSSEINIDOUST, Hamilton (CA); Tohid DIDAR, Hamilton (CA); Fereshteh BAYAT, West Hamilton (CA)

(21) Appl. No.: 19/490,116

(22) PCT Filed: Jun. 6, 2024
(86) PCT No.: PCT/CA2024/050758

§ 371 Date: Dec. 4, 2025

Related U.S. Application Data
(60) Provisional application No. 63/471,334, filed on Jun. 6, 2023.

(51) Int. Cl. C12Q 1/18; C12N 11/10; C40B 30/06

(52) U.S. Cl. CPC C12Q 1/18; C12N 11/10; C40B 30/06

(57) ABSTRACT

A composition comprises a sugar-based matrix, wherein the sugar-based matrix encapsulates a phage and a reagent for detecting a bacterial cell condition. In aspects, the bacterial cell condition is cell growth, cell metabolic activity, cell death, cell membrane integrity, cell lysis, or any combination thereof. Related screening platforms, libraries, and methods are also described.

The present disclosure relates to the field of antimicrobial resistance, and in particular, to bacteriophage biobanks, compositions, and related methods.

### BACKGROUND

Phage therapy, viral phage therapy, or phagotherapy is the therapeutic use of bacteriophages for the treatment of pathogenic bacterial infections. This therapeutic approach emerged at the beginning of the 20th century but was progressively replaced by the use of antibiotics in most parts of the world after the Second World War. Bacteriophages, known as phages, are a form of virus that attach to bacterial cells and inject their genome into the cell. The bacteria's production of the viral genome interferes with its ability to function, halting the bacterial infection. The bacterial cell causing the infection is unable to reproduce and instead produces additional phages. Phages are very selective in the strains of bacteria they are effective against.

The plaque assay is a critical tool for phage isolation and quantitation. The number of infectious phage particles in a sample is measured in ‘plaque-forming units’ or pfu. Titration of a phage suspension is carried out by mixing serial dilutions of the sample with aliquots of sensitive bacteria. This mixture is plated in a soft agar overlay on a petri dish containing the appropriate nutrient agar and incubated, usually overnight. Individual infected cells release phages that infect the surrounding cells in the bacterial layer, and multiple rounds of infection continue until growth of the bacteria on the plate ceases due to nutrient depletion. At suitable phage dilutions, this will lead to the appearance of individual holes, or plaques, in an otherwise confluent bacterial lawn. Since each plaque is derived from a single viable phage, counting the number of plaques allows calculation of the number of plaque-forming units in the original suspension.

U.S. Patent Application Publication No. 20220065851 is directed to methods of performing chemical reactions, including multi-step chemical reactions in which two or more of the reagents in the chemical reaction are incorporated or entrapped in a solid polymeric structure comprising pullulan. In certain embodiments, the chemical reaction or multi-step reaction serves as a sensor. That application is also directed to sensors for performing the methods. In certain embodiments, at least one of the reagents is a biomolecule and the sensor is a biosensor. In certain other embodiments, the solid polymeric structure comprising pullulan and the reagents for performing a chemical reaction form a convenient device for performing a chemical reaction.

The background herein is included solely to explain the context of the disclosure. This is not to be taken as an admission that any of the material referred to was published, known, or part of the common general knowledge as of the priority date.

### SUMMARY

In accordance with an aspect, there is provided a composition comprising a sugar-based matrix, wherein the sugar-based matrix encapsulates a phage and a reagent for detecting a bacterial cell condition.

In an aspect, the bacterial cell condition is cell growth, cell metabolic activity, cell death, cell membrane integrity, cell lysis, or any combination thereof.

In an aspect, the bacterial cell condition is bacterial cell lysis.

In an aspect, the sugar-based matrix comprises a sugar with a Tg of greater than about 40 degrees C.

In an aspect, the sugar-based matrix comprises a sugar without reducing groups.

In an aspect, the sugar-based matrix comprises an oligosaccharide and a disaccharide sugar.

In an aspect, the sugar-based matrix comprises a sugar that is a desiccant.

In an aspect, the sugar comprises trehalose.

In an aspect, the sugar-based matrix comprises pullulan, maltodextrin, dextran, inulin, alginate, cellulose, trehalose, sucrose, lactose, maltose, or any combination thereof.

In an aspect, the sugar-based matrix comprises pullulan and trehalose.

In an aspect, the reagent detects ATP and/or XTT.

In an aspect, the reagent comprises XTT tetrazolium salt (2,3-Bis-(2-Methoxy-4-Nitro-5-Sulfophenyl)-2H-Tetrazolium-5-Carboxanilide) and menadione.

In an aspect, the composition is stable at room temperature for at least four weeks under vacuum.

In an aspect, the composition demonstrates susceptibility profiling at low MOI (low efficacy of plaquing).

In an aspect, the bacterial cell condition is detected through a colour change, fluorescence, and/or bioluminescence.

In an aspect, the generated bioluminescence signal in increased by up to about 90% as compared to a composition without the sugar-based matrix.

In an aspect, the composition provides for phage susceptibility screening in less than about 120 minutes, such as about 30 mins.

In an aspect, the sugar-based matrix enhances desiccation tolerance of the phage and/or the reagent.

In an aspect, the composition is in solid or gel form, such as a tablets, pill, disc, or is printed or coated on a surface.

In accordance with an aspect, there is provided a combination comprising (1) a composition comprising a sugar-based matrix, wherein the sugar-based matrix encapsulates a phage, and (2) a reagent for detecting a bacterial cell condition.

In accordance with an aspect, there is provided a high throughput phage screening platform comprising a plurality of the compositions described herein, wherein each composition comprises a different phage.

In an aspect, the platform comprises a multi-well plate, a portable chip, a microfluidic device, a lateral flow device, a printed microarray, or a solid film.

In accordance with an aspect, there is provided a high throughout phage screening platform comprising a portable library of individual shelf-stable, ready-to-use phages, in solid tablets, wherein the tablets optionally contain reagents for detecting a bacterial cell condition.

In accordance with an aspect, there is provided a method for detecting phage-mediated bacterial cell lysis, the method comprising applying a bacterial culture to the composition, combination, or platform described herein.

In accordance with an aspect, there is provided a method for developing and preserving bacteriophage biobanks for one-pot phage susceptibility screening, the method comprising:
a) preparing the one-pot phage susceptibility screening assay, comprising combining in a solid or gel form: i) one or more different bacteriophage strains ii) reagents required for biochemical assays detecting products of phage-mediated bacterial lysis optionally, iii) a sugar polymer matrix;
b) applying one or more samples comprising multi-drug resistant bacterial suspensions to the one-pot phage susceptibility screening assay;
wherein generation of a detectable signal upon applying one or more samples indicates susceptibility of the multi-drug resistant bacterial suspension to the one or more bacteriophages present in the assay.

In an aspect, the sugar polymer matrix comprises pullulan and/or trehalose.

In an aspect, the sugar polymer matrix provides heat stability and protects against desiccation for the one or more different bacteriophage strains and reagents required for biochemical assays, allowing for storage and easy transport without refrigeration.

In an aspect, the combination of one or more different bacteriophages, reagents required for biochemical assays detecting products of phage-mediated bacterial lysis and, optionally, sugar polymer matrix are in a solid or gel form comprising but not limited to tablets, pills, discs, or printed or coated on a surface.

In an aspect, the detectable signal comprises bioluminescent, colorimetric, or fluorescent signals.

In an aspect, the reagents required for biochemical assays detecting products of phage-mediated bacterial lysis comprise ATP bioluminescence assay reagents, XTT assay reagents.

In an aspect, the generation of a signal can be detected in as little as 30 mins.

In an aspect, bacteriophage susceptibility screening can be completed in single or multi-well plates, portable chips, microfluidic devices, lateral flow devices, printed microarrays, or on a solid film for high throughput and/or low throughput applications.

In an aspect, the one or more samples containing bacteria comprise biological samples, environmental samples, and/or clinical samples.

In an aspect, phages that have been identified to lyse the target bacteria strain(s) are used for applications comprising human and/or animal phage therapy, environmental biocontrol, food chain decontamination, human supplements, and/or animal farming.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1. Schematic of vision and phage characterization. a Personalized phage therapy. The first step in personalized phage therapy is the isolation of treatment-resistant bacterial strain from patient (i), followed by employing slow culture-based methods to screen and select therapeutic phages (ii). We propose a single-tablet technology for rapid, high-throughput screening of therapeutic phages (iii). b Phage-mediated ATP release and detection. Phage-mediated lysis of bacterial cell starts when a phage virion encounters the host cell and attaches to specific phage receptors (i), this is followed by phage genome entry into the host bacteria (ii). This starts a cascade of events leading to hijacking of bacteria replication machinery for synthesis of new progeny phage (iii), which ultimately leads to host cell lysis (iv), and release of progeny virions along with a burst of ATP (v). ATP reacts with luciferin to form luciferin adenylate, which is oxidized by the luciferase enzyme in the presence of magnesium to form oxyluciferin, CO₂ and adenosine monophosphate (AMP), which results in light emission (vi). c Transmission electron micrographs of three phages namely P32, JG004 and PP7. d Plaque formation on Pa lawns created by P32, JG004 and PP7. e Metabolic activity of uninfected and infected (MOI=10, 1, 0.1, 0.01, 0.001) Pa cultures (n=3, mean±SD). All reported values are the mean of three biological replicates and associated error bars shown as dashed lines represent standard deviation from the mean.

FIG. 2. Optical density (OD₆₀₀) or growth curve of Pseudomonas aeruginosa PAO1 strain (Pa). a Pa infected with P32 shows a strong performance in preventing bacterial growth by decreasing the culture turbidity even at lower MOIs (0.001). b Pa infected with JG004 showed effective bacterial growth suppression c Pa infected with PP7 showed week efficacy is preventing bacterial growth which aggravates by decreasing the initial concentration of phage (lower MOIs). The represented data are the average of three independent experiments (n=3) with at least 6 technical replicates.

FIG. 3. Schematics of phage life cycles. a Lytic life cycle. The lytic cycle of phages starts with (i) phage recognition and attachment to bacterial cell receptors, (ii) genome injection, (iii) taking control of the bacterial replication machinery and producing new virions, and (iv) release of progeny phages by bacterial cell lysis. b Lysogenic life cycle. In the lysogenic life cycle, phage also adsorbs to cell receptors (i) and injects its genome inside the bacterial cell (ii); however, the phage genome is integrated into the bacterial genome, forming a prophage (iii) that is replicated through the bacterial reproduction cycle (iv). The lysogenic cycle could be induced to start the lytic cycle with environmental triggers such as high temperature or UV exposure which can ultimately lead to cell lysis (as shown by the dashed arrow). c Chronic life cycle. Similar to other phage life cycles, the chronic lifestyle (mostly seen in filamentous phages) starts with recognition and adsorption to bacterial cell receptors (i) and genome injection (ii). By taking control of the bacterial replication machinery, new virions are synthesized inside the bacterial cell (iii) and are released through budding or extrusion without lysing bacterial cells (iv). Some chronic phages can also adopt a lysogenic lifestyle, in which the phage genome is incorporated into the bacterial genome forming a prophage (iii′) and is replicated through the bacterial reproduction cycle and staying dormant (iv′) until the chronic cycle is induced by environmental stimuli (shown by the dashed arrow).

FIG. 4. Decreasing ATP background in phage solutions. a ATP background at different stages of P32 phage purification. PEG purification decreased the ATP background in phage solution. 10 KDa ultrafiltration decreased the ATP background significantly, while 3 KDa filters failed to reduce ATP levels. b Effect of dilution of phage suspension in bacterial cell culture media (LB media) on ATP background. The effect of diluting phage samples is comparable to purifying phage suspensions. Depending on the availability, either diluted filtered phages in bacterial cell media/buffer or purified phages can be used to reduce the effect of the background bioluminescence signal intensity. Results shown are the average of three replicates (n=3) with associated error bars showing standard deviation from the mean.

FIG. 5. Sequential assay for detection of phage-mediated ATP release. a Workflow schematic: Bacterial cultures were infected with phages at different concentrations (i) and incubated at 37° C. (ii), at different time intervals, ATP was measured at room temperature by adding ATP reagent solution which contains luciferin and luciferase as main components (iii), and bioluminescence signal was measured (iv). Concentration of ATP was calculated for bioluminescence signal measured from Pa individually infected with P32, JG004, and PP7 at different MOIs: b MOI=10, c MOI=1, d MOI=0.1, e MOI=0.01, f MOI=0.001. g time to signal for Pa infected with P32 and JG004 at different MOIs. All reported values are the mean of three independent experiments with n=3 and associated error bars represent standard deviation from the mean. Statistical significance in all panels is derived from Two-way analysis of variance (ANOVA). Significance levels include *P<0.05, ** P<0.01, *** P<0.001, and **** P<0.0001.

FIG. 6. One-pot ATP bioluminescence assay. a Workflow schematic. ATP one-pot assay includes adding all ATP bioluminescence assay reagents (luciferin and luciferin as major components) and phage, followed by adding bacterial suspension and incubating the plate at 37° C. for real-time measurement of phage-induced ATP release. b Kinetic ATP measurement for Pa infected with P32, JG004, and PP7 at MOI=10 and uninfected Pa. (n=3, and the dashed line shows standard deviation from the mean). c Time to peak signal for P32 and JG004 at different MOIs (n=3). d Signal at peak for Pa cultures infected with P32, JG004, and PP7 at different MOIs including MOI=10, MOI=1, MOI=0.1, MOI=0.01, MOI=0.001. All reported values are the mean of three independent experiment with n=3, and associated error bars represent standard deviation from the mean. Statistical significance in d is derived from one-way analysis of variance (ANOVA). Significance levels include Significance levels include *P<0.05, ** P<0.01, *** P<0.001, and **** P<0.0001.

FIG. 7. One-pot ATP assay in the presence of stabilizing sugar polymer matrix. a Workflow schematic. Bacterial suspensions are added to 96 well plate containing phages mixed with ATP reagents in sugar mixture. b Bioluminescence signal at peak for Pa cultures infected with phages in the presence versus absence of sugar mixture in fresh liquid one-pot ATP assay. c Bioluminescence signal at peak and assay end point of 6 hours for JG004 infected Pa cultures in the presence versus absence of sugar mixture in fresh liquid one-pot ATP assay JG004. d Continuous measurement of bioluminescence signal for Pa infected with P32, JG004, or PP7 in fresh liquid sugar mix solutions. e Kinetic measurement of bioluminescence signal for Pa infected with P32, JG004, and PP7 in reconstituted one-week old tablets. f Kinetic measurement of bioluminescence signal for Pa infected with P32, JG004, and PP7 in rehydrated phage and enzymes dried in the absence of sugar polymers showing complete loss of signal after one week storage. g Same data as f but with a narrower y-axis scale, clearly showing the loss of signal. Error bars in b and c graphs show the statistical analysis based on unpaired t-test, associated error bars represent standard deviation from the mean. Significance levels include *P<0.05 and ** P<0.01. Dashed line in d, e, and g show standard deviation from the mean of three independent experiments.

FIG. 8. Effect of sugar polymers on stability of the ATP reagent solution at 37° C. ATP standard solutions were added to ATP reagent solution with (+) and without (−) sugar mixture at time =0, after incubating for one, three and six hours at 37° C., and the results were compared to no heat exposure ATP reagent solution. ATP standard solutions are used at a 0.4 μM, b 0.01 μM, and c 0.001 μM. The RLU signal was measured kinetically every 10 minutes for up to 3 hours. Sugar mixture was able to preserve the activity of luciferin and luciferase as there were no significant changes in the RLU signals after heat exposures. In the absence of sugars, the activity of ATP reagent solution decreased as significantly lower RLU signals were detected, which was more drastic after 3 and 6 hours of heat exposure. The RLU signal also dropped faster in the absence of sugar mixture. The presented data are the average of three replicates (n=3) with error bars representing standard deviation from the mean.

FIG. 9. Comparison of the RLU signal intensity of heat treated and untreated ATP reaction solutions in the presence and absence of sugar mixture after heat exposure at 37° C. RLU signal at time=0, right after adding ATP standard solution with 0.4 μM, 0.01 μM, 0.001 μM to ATP reagent solutions with (+) and without (−) sugar mixture after incubating for 1, 3 and 6 hours at 37° C. in comparison with no heat exposure ATP reagent solution (time=0). The presented data are the average of three replicates (n=3) with Standard deviation from the mean. Statistical significance in all panels is derived from Two-way analysis of variance (ANOVA). Significance levels include *P<0.05, ** P<0.01, *** P<0.001, and **** P<0.0001.

FIG. 10. Stability of dried Pseudomonas phages P32 (a), JG004 (b), and PP7 (c) in ambient conditions in 10 wt % pullulan+0.5 M trehalose in comparison with no sugars. Sugar polymers helped to retain higher infectivity after 30 days. The presented data are the average of three replicates (n=3) with Standard deviation from the mean.

FIG. 11. Phage and ATP reagents stability assay after 4 weeks. The all-inclusive tablets including phages and ATP reagent solutions in sugar polymer matrices were stored for 4 weeks at room temperature under vacuum. To run the assay, the tablets were reconstituted with sterile Milli-Q water, followed by addition of P. aeruginosa (Pa) subcultures at OD₆₀₀ ~0.1 and kinetic monitoring of RLU signal. The data presented are the average of 6 replicates, and dashed line showing standard deviation from the mean.

FIG. 12. Large-scale phage library screening of stabilized phage biobank. a Proposed workflow for screening a bacterial isolate against a phage library using our platform technology, where the phage library is screened by detecting phage-mediated ATP release using ATP bioluminescence assay in the presence of sugar polymers stabilizers. The assay can be conducted with fresh liquid sugar-polymer based ATP reagent solution or conducted on reconstituted all-inclusive sugar-based tablets. b The urinary tract infection isolate, P. aeruginosa C0072 and c the arm infection isolate, P. aeruginosa C0335 were screened against an in-house library of all-inclusive tablets. Bioluminescence signal was recorded every 5 minutes and the snapshot of the signal at time=60 min is shown for each well. d The sewage isolate, S. enterica serovar Newport and e the human blood isolate, S. enterica serovar Senftenberg were screened against a library of Salmonella phages containing 16 phages (listed in Table 2) in the presence of sugar polymer stabilizers. The presented data are the snapshots at 90 min time point for both Salmonella strains. f The fecal isolate, E. coli 0157:H7 and g The Crohn's disease isolate, E. coli LF82 were screened against a library of E. coli phages containing 28 E. coli phages listed in Table 3 in the presence of sugar polymer stabilizers. The presented data are the snapshot at 100 min time point for both E. coli strains. h The snapshot of data at time=170 min for S. aureus 68 strain and g the snapshot at time=100 min for S. aureus 44A strain screened against a library of S. aureus phages containing 9 phages (listed in Table 4). Data points represent 3 replicates.

FIG. 13. Screening Clinical P. aeruginosa isolates against an in-house phage library. a C0072 strain isolated form patient with urinary tract infection screened against a library of phages including phages from different species (Table 1) using desiccated Sugar-based one-pot ATP bioluminescence assay, optical density assay (OD₆₀₀), and representative spot test on C0072 bacterial lawn. b C0335 strain isolated from patient with arm infection, screened against an in-house phage library using one-pot ATP bioluminescence assay optical density assay (OD₆₀₀) and spot test on C0335 bacterial lawn. The OD₆₀₀ assay and spot test was conducted on three P. aeruginosa phages including P32, JG004, and PP7. ATP and OD assay were conducted with at least three replicates (n=3).

FIG. 14. Metabolic activity measurement of clinical isolates of P. aeruginosa with P32, JG004, and PP7 at MOI ~10. a Metabolic activity of phage infected C0335. There were no significant changes in metabolic activity of phage infected C0335 compared to uninfected C0335. b Metabolic activity of phage infected C0072. metabolic activity of C0072 decreased after 2 hours for C0072 infected with P32. The data presented are the average of at least 3 replicates (n=3).

FIG. 15. Zoomed in image of the phage library screening, showing a delayed increase in bioluminescence signal for C0035 infected with JG004 appearing after 2 hours.

FIG. 16. ATP and OD600 kinetic curves and spot test of two Salmonella strains against 16 Salmonella phages. a Salmonella enterica serovar Newport (S. enterica serovar Newport). b Salmonella enterica serovar Senftenberg (S. enterica serovar Senftenberg). ATP one-pot assay was conducted in the presence of sugar polymers. The ATP and OD₆₀₀ were conducted in triplicates (n=3), spot tests were repeated independently twice. The error bars show standard deviation from the mean.

FIG. 17. ATP and OD600 kinetic curves and spot test of two E. coli strains against 28 E. coli phages. a E. coli O157:H7 strain (human fecal isolate). b LF82 (Crohn's disease isolate¹). ATP one-pot assay was conducted in the presence of sugar polymers. The ATP and OD₆₀₀ were conducted in triplicates (n=3).

FIG. 18. ATP and OD₆₀₀ kinetic curves and spot test of two S. aureus strains against nine S. aureus phages. a HER 1049 S. aureus 68 strain. b HER 1101 S. aureus 44A. ATP one-pot assay was conducted in the presence of sugar polymers. The ATP and OD₆₀₀ were conducted in triplicates (n=3).

FIG. 19. Infecting P. aeruginosa strains with phage JG004 phage at different MOIs. Original phage JG004 titer was ~10¹¹ PFU/mL, which was diluted and spotted on the bacteria lawns. Dilution factors are shown on spot test plates ranging from 1 to 10⁻⁸(~5×10¹¹ PFU/mL to ~5×10³ PFU/mL). For the ATP and OD₆₀₀ assays, bacterial subcultures were infected with phage JG004 at MOIs of 10,000, 1000, 100, 10, 1, 0.1, 0.01. Panel a) Pa: JG004, b) C0072: JG004, c) C0335: JG004.

FIG. 20. Flowchart illustrating the overall time needed for susceptibility screening with various methodologies, namely spot test, kill curve, and the new proposed method, for a library of ~100 phages and one person dedicated to the task. Created with BioRender.com

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “one-pot”, as used herein refers to the set of successive chemical reactions in which all reagents are added into one vessel to increase efficiency of a chemical reaction.

It will be understood that any component defined herein as being included may be explicitly excluded by way of proviso or negative limitation, such as any specific compounds or method steps, whether implicitly or explicitly defined herein.

### II. Compositions and Methods

In certain aspects, an ultraportable, high throughput phage screening platform is provided in a shelf-stable, ready-to-use, all-inclusive solid or gel format, eliminating the requirement for stage-wise assays where phage libraries must be refrigerated or frozen. Phage or phages along with the biochemistry of detection are encapsulated in solid or gel format, stabilized in a matrix of sugar polymers that confer heat stability to detection enzymes, increasing the generated detectable signal by ~90% and allowing for phage susceptibility screening in as low as 30 mins, as well as enhancing desiccation tolerance of all components, providing easier and cheaper transportation of this technology around the world and as a result, increased accessibility to therapeutic phage. This technology is a paradigm shift from the century-old, slow, and labour-intensive plaque assay, offering the promise of enhanced accessibility and potentially lowering mortality rates for patients with antibiotic-resistant infections through rapid personalized phage therapy.

Thus, provided herein is a composition comprising a sugar-based matrix. The sugar-based matrix encapsulates a phage. Typically, the sugar-based matrix also encapsulates a reagent for detecting a bacterial cell condition. The reagent can detect the cell condition directly or indirectly, however, typically the cell condition is detected directly. In other words, typically the reagent detects a cell mediator or metabolite for example that is immediately linked to the condition being analyzed rather than a proxy for the condition.

While the reagent is typically included in the sugar-based matrix with the phage, it will be understood that the reagent can be provided separately as desired and understood by the skilled person. For example, combinations or kits comprising the encapsulate phage together with the reagent being provided in a separate vessel are contemplated. Conveniently these are typically provided in a single composition, however, separate provision is also contemplated.

Thus, in aspects, there is provided a composition comprising a sugar-based matrix, wherein the sugar-based matrix encapsulates a phage and a reagent for detecting a bacterial cell condition.

Any cell condition that can be detected using a reagent as an indicator is contemplated. Typically, the bacterial cell condition relates to life, death, or metabolism of a bacterial cell. For example, in aspects, the cell condition is cell growth, cell metabolic activity, cell death, cell membrane integrity, cell lysis, or any combination thereof. Typically, lytic phages are being sought after in the methods contemplated herein and, therefore, typically, the cell condition is bacterial cell lysis.

The sugar-based matrix may comprise any sugar or combination of sugars. Sugars can be categorized into mono-, di-, oligo-, and polysaccharides. Monosaccharides like glucose, fructose, and galactose are not typically suitable as protein stabilizers during storage since these sugars have a low Tg (<40° C.) and contain reducing groups.

The disaccharides sucrose and trehalose possess a much higher Tg (77° C. for sucrose and 121° C. for trehalose₁₅) and do not contain reducing groups and are therefore good choices as protein stabilizers.

A downside of many high molecular weight sugars is the combination of a large size with the limited flexibility of the molecular chains. Due to this combination, hydrogen bond interactions with proteins are sterically hindered and efficient vitrification at the surface of the protein will become difficult to achieve.

An advantage of oligosaccharides like inulin and dextran is their high Tg values (the Tg values of moisture free inulin with an average molecular weight of 4 kDa and dextran 5 kDa are 157 and 176° C., respectively).

For this reason, addition of a polysaccharide with a high Tg (like pullulan) to the disaccharide (like trehalose) has been shown herein to provide a benefit by combining a proper coating with a high Tg.

Thus, in typical aspects, the sugar-based matrix comprises a sugar with a Tg of greater than about 40 degrees C., such as greater than about 40, 45, 50, 55, 60, 65, 70, 75, 80, 85, 90, 95, 100, 105, 110, 115, or 120 degrees C. Examples include pullulan, maltodextrin, dextran, inulin, alginate, and cellulose.

Similarly, in typical aspects, the sugar-based matrix comprises a sugar without reducing groups. Examples include trehalose, sucrose, lactose, maltose.

Typically, the sugar-based matrix comprises an oligosaccharide and a disaccharide sugar, such as pullulan, maltodextrin, dextran, inulin, alginate, cellulose, trehalose, sucrose, lactose, maltose, or any combination thereof.

In some aspects, the sugar-based matrix comprises a sugar that is a desiccant, such as trehalose. In this way, the sugar-based matrix in aspects enhances desiccation tolerance of the phage and/or the reagent.

Typically, the sugar-based matrix comprises pullulan and trehalose.

With respect to the reagent or reagents included in the composition for detecting a cell condition, the skilled person is aware of such reagents and assays that are typically used to detect any particular cell condition, such as measures of life, death, or cell metabolism. Typically, the reagent detects ATP and/or XTT as indicators of cell lysis. For example, when XTT is used, the the reagent comprises XTT tetrazolium salt (2,3-Bis-(2-Methoxy-4-Nitro-5-Sulfophenyl)-2H-Tetrazolium-5-Carboxanilide) and menadione.

Due to the inclusion of the sugar-based matrix, the composition described herein is surprisingly stable at room temperature. For example, in aspects, the composition is stable for at least 2, 3, 4, 5, 6, 7, 8, 9, 10, 11, or 12 weeks under vacuum. Furthermore, advantageously, the composition described herein demonstrates susceptibility profiling at low MOI (low efficacy of plaquing).

The bacterial cell condition can be detected through any known method. For example, there may be a colorimetric change that is visible to the naked eye or detectable by machine. Alternatively, the condition may be detected through fluorescence, and/or bioluminescence. In typical aspects, the generated bioluminescence signal in increased by up to about 90% as compared to a composition without the sugar-based matrix.

It will be understood that, advantageously, the compositions described herein provide a relatively fast screening tool. For example, in aspects, the composition provides for phage susceptibility screening in less than about 120 minutes, such as less than about 120, 110, 100, 90, 80, 70, 60, 50, 40, or 30 minutes, such as about 30 mins.

The composition is typically dried or concentrated so as to be provided in solid or gel form, such as a tablet, pill, disc, or is printed or coated on a surface. For example, typically methods of drying may include casting glass sugar films, freeze dried powder, or spray dried formulation.

The concentration of the sugar can vary based on the chosen drying method, generally from about 1 to about 35% w/v. For example, for spray drying, lower concentrations in the range 1-10% w/v, for freeze drying and film/tablet casting, medium (10-20% w/v) or high (20-35% w/v) are typical.

Also provided herein is a high throughput phage screening platform or phage library comprising a plurality of the compositions described herein, wherein each composition comprises a different phage. In aspects, there is provided a high throughout phage screening platform comprising a portable library of individual shelf-stable, ready-to-use phages, in solid tablets, wherein the tablets optionally contain reagents for detecting a bacterial cell condition. The platform is provided in any known method for high throughput screening. For example, typically it comprises a multi-well plate, a portable chip, a microfluidic device, a lateral flow device, a printed microarray, or a solid film.

Methods are also described herein, for making the compositions, assembling the libraries, and for detecting a cell condition, such as phage-mediated bacterial cell lysis. The method typically comprises applying a bacterial culture to the composition, combination, or platform described herein.

In some aspects, there is provided a method for developing and preserving bacteriophage biobanks for one-pot phage susceptibility screening, the method comprising:
a) preparing the one-pot phage susceptibility screening assay, comprising combining in a solid or gel form: i) one or more different bacteriophage strains ii) reagents required for biochemical assays detecting products of phage-mediated bacterial lysis optionally, iii) a sugar polymer matrix;
b) applying one or more samples comprising multi-drug resistant bacterial suspensions to the one-pot phage susceptibility screening assay;
wherein generation of a detectable signal upon applying one or more samples indicates susceptibility of the multi-drug resistant bacterial suspension to the one or more bacteriophages present in the assay.

The one or more samples containing bacteria can be from any source. For example, typically, the samples comprise biological samples, environmental samples, and/or clinical samples.

Similarly, phages that have been identified to lyse the target bacteria strain(s) are typically used for applications comprising human and/or animal phage therapy, environmental biocontrol, food chain decontamination, human supplements, and/or animal farming.

### EXAMPLES

The following non-limiting examples are illustrative of the present disclosure:

### Example 1: High Throughput Platform Technology for Rapid Target Identification in Personalized Phage Therapy

### Abstract

As bacteriophages continue to gain regulatory approval for personalized human therapy against antibiotic-resistant infections, there is a need for transformative technologies that address century-old challenges, specifically for rapid target identification through multiple, large, decentralized therapeutic phages libraries. Combining materials technology and biochemistry, we designed a high throughput phage screening platform comprised of a portable library of individual shelf-stable, ready-to-use phages, in all-inclusive solid tablets. Each tablet encapsulates a phage along with the biochemistry for detection of cell lysis, stabilized in a matrix of sugar polymers that confer stability, increasing the generated bioluminescence signal by ~90% and allowing for phage susceptibility screening in as low as 30 mins. The tablet composition also enhances desiccation tolerance of all components, enabling easier and cheaper international transportation of phages and as a result, increased accessibility to therapeutic phages. High throughput screening was demonstrated by stabilizing an in-house, referenced phage biobank in the form of in all-inclusive solid tablets and identifying target phages for select multidrug-resistant clinical isolates of Pseudomonas aeruginosa, Salmonella enterica, Escherichia coli, and Staphylococcus aureus with targets identified in 30 to 100 min. This technology is a paradigm shift from slower and labour-intensive culture techniques, offering the promise of enhanced phage accessibility for patients with antibiotic-resistant infections.

### Introduction

The spread of antimicrobial resistance (AMR) is considered a major global challenge, claiming more than 700,000 lives per year, with a projected increase to 10 million by 2050.¹ The looming global crisis of AMR and urgency to identify new therapeutics to fight against acute and chronic bacterial infections has garnered the attention of researchers, clinicians, and regulatory bodies towards bacteriophage therapy (i.e., the use of bacteriophages to fight bacterial infections). Bacteriophages, or phages for short, are viruses that exclusively infect bacteria in a highly targeted manner.² This targeted killing action is in stark contrast to the indiscriminate action of antibiotics and is a major advantage of phages over antibiotics, promising minimal disruption to our microbiota,³⁻⁵ while simultaneously challenging decades of experience in development of antimicrobial therapies.

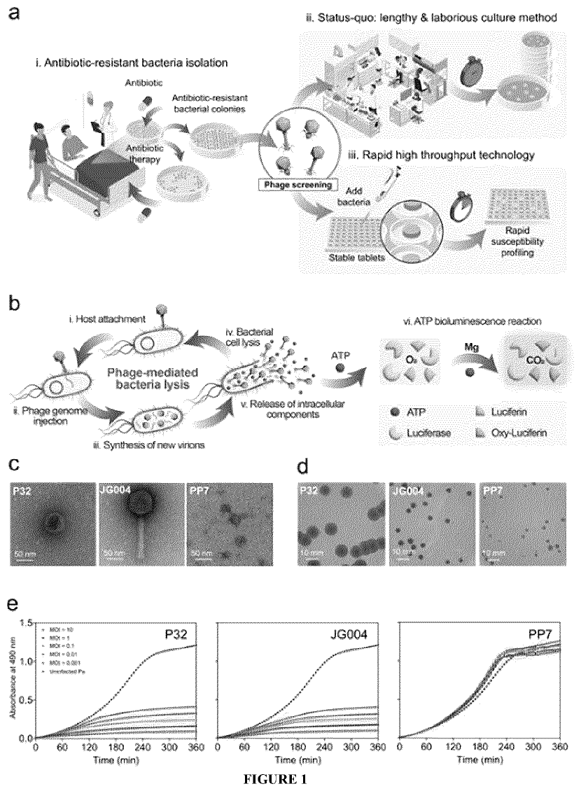

Phage therapy often relies on labor-intensive and time-consuming methods that may fall short when a human life hangs in the balance. Design and administration of phage therapeutics requires a fundamental shift in the way we think about antimicrobial therapies. Among others, there is a need for custom-designed tools and technologies that meet the standard of care in modern medicine. One of the challenges in clinical practice of phage therapy is related to the targeted action of phages. Indeed human phage therapy is most effective when implemented as a personalized therapy, as demonstrated by an increasing number of clinical case reports.⁵⁻⁷ Personalized phage therapy begins with the isolated pathogenic bacterial strain deemed to be resistant to available antibiotics and its screening against large libraries of therapeutic phages and/or libraries of environmental samples expected to contain phages (FIG. 1a-i). These libraries can contain hundreds of phages and thus require susceptibility profiling technologies that can be implemented rapidly and in a high throughput format. The current gold standard in susceptibility testing, is the spot test,⁸ a culture-based method which involves a long incubation period of overnight to several days depending on the targeted bacteria (FIG. 1a-ii).⁹ Although widely used in research labs, the spot test method is laborious and slow. In the common spot tests, as a result of phage lysing bacterial cells and the release of progeny phages, clear zones are formed on solid medium⁹. Liquid assays are also performed to monitor bacterial lysis as a result of phage infection and they include tracking the bacteria culture turbidity (optical density) as a result of phage lysis¹⁰. The main hurdle of monitoring optical density of a bacteria culture is that bacteria cell debris can contribute to turbidity of the culture and affect the results negatively.¹¹ The overall metabolic activity of phage-infected bacterial cultures can also be monitored using various biochemical assays that have gained much attention recently and can be used to analyze properties related to phenotypes, namely cell growth and respiration.¹¹ However, these metabolic assays are detecting the phage-mediated bacterial cell lysis indirectly.

The second challenge is that a universal phage library is nonexistent and patients, researchers, and clinicians pursuing phage therapy depend on limited libraries maintained by local and, in very few cases, national or military research labs.³ Successful implementation of personalized phage therapy therefore requires streamlined communication and sharing between phage libraries at the national and international levels.¹² We envision a platform that addresses both aforementioned challenges in personalized phage therapy, namely a reliable and rapid high throughput technology with shelf-stable and portable assay reagents that can be readily shipped around the world to be employed at the point of care with minimal infrastructure or training. To meet these design criteria, we re-imagined a therapeutic phage library in the physical format of stable and all-inclusive solid tablets, each tablet encapsulating a single phage along with the biochemistry to detect phage-mediated cell lysis (FIG. 1a-iii).

Here, we explore the rapid detection of released adenosine triphosphate (ATP) as a proxy for phage-mediated bacterial lysis (FIG. 1b). To do, we needed to overcome two major barriers in the biochemistry of ATP detection. ATP can be readily detected with firefly luciferase,¹³ which is a heat labile enzyme and quickly deactivated at temperatures above 30° C.¹⁴ For human therapeutic applications, however, lytic activity of bacteriophages must be detected at 37° C., which is optimal temperature for growth and metabolic activity of clinical bacterial pathogens and thus for phage lytic action. In addition, commercially available ATP detection assays require stagewise addition of reaction components, complicating high throughput implementation.

To overcome these barriers in detection, we utilized materials technology to stabilize and encapsulate all reagents required for ATP detection along with each phage in a sugar-based matrix that prevented thermo-inactivation of the enzymes. Optimizing the biochemical rection in the presence of this matrix enabled realization of a hassle-free, one-pot biochemical reaction, stabilized in the form of a solid tablet that preserved the activity of the enzymes at physiological temperatures as well as phage infectivity. The latter promises easier and cheaper transportation and as a result, easier screening of decentralized phage libraries and increased accessibility to phage therapy. We demonstrated the utility of this platform technology for high throughput implementation by phage susceptibility screening of selected isolates of Pseudomonas aeruginosa (Pa), Salmonella enterica, Escherichia coli, and Staphylococcus aureus against in-house phage libraries.

### Results

Phage-mediated bacteria lysis. To develop a one-tablet assay based on detection of phage-mediated lysis, we started by selecting three phages from our in-house phage library with different levels of bacteria lysis ability. Bacteriophages vB_Pae-Tbilisi32 (P32), JG004, and PP7 were propagated using our host bacterial strain, P. aeruginosa PAO1 (Pa). Transmission Electron micrographs of the three phages are shown in FIG. 1c. P32 is a podophage and has a very short tail,¹⁵ JG004 is a myophage¹⁶ with an isometric head and a contractile tail. Both of these phages have a double-stranded DNA genome. Phage PP7 belongs to Leviviridae family (single-stranded RNA genome) and has an icosahedral capsid with an approximate diameter of 30 nm (FIG. 1c).¹⁷

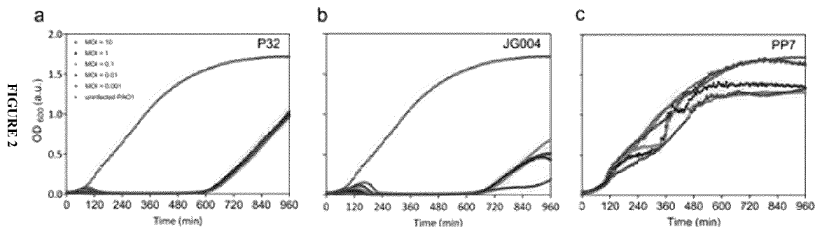

FIG. 1d also shows plaques generated by each of the three phages on Pa bacterial lawn, indicative of the ability of these phages to successfully infect and lyse Pa.^{18,19} Plaque morphology can sometimes provide qualitative yet important information regarding phage characteristics. For example, larger plaques may be indicative of a larger burst size (number of progeny phages released from the infection of a single bacterial cell)²⁰ and shorter latent period (period between phage adsorption and release of progeny virions).²¹ As seen in FIG. 1d, P32 generates the largest plaque, followed by JG004, and then PP7. To further characterize phage-mediated bacteria lysis, we generated kinetic kill curves which showed the change in metabolic activity of the bacterial culture challenged with the three phages, using the XTT colorimetric assay (FIG. 1d). Pa was infected at different MOIs (multiplicity of infection, defined as the ratio of infectious virions to bacterial cells in a culture).²² For all experiments, starting concentration of bacteria was kept constant (~10⁷ CFU/mL). Both phages P32 and JG004 (FIG. 1d) significantly suppressed bacterial growth, as indicated by a low metabolic activity. The decrease in bacterial metabolic activity was slower at lower MOIs, as expected. It is noteworthy that although PP7 formed visible plaques and lysis zone on solid medium, it did not suppress bacterial growth in our liquid assays. (FIG. 1d). These trends agree with Pa kill curves based on optical density (FIG. 2) and highlight a very important bias of standard methods in phage susceptibility testing, specifically as it pertains to phage application for therapy and biocontrol. Indeed, a clearing on a bacterial lawn (plaque or spot test) does not necessarily signal the ability of a phage to control the population of bacteria in a liquid culture, and as some studies have indicated, in in vivo models.²³

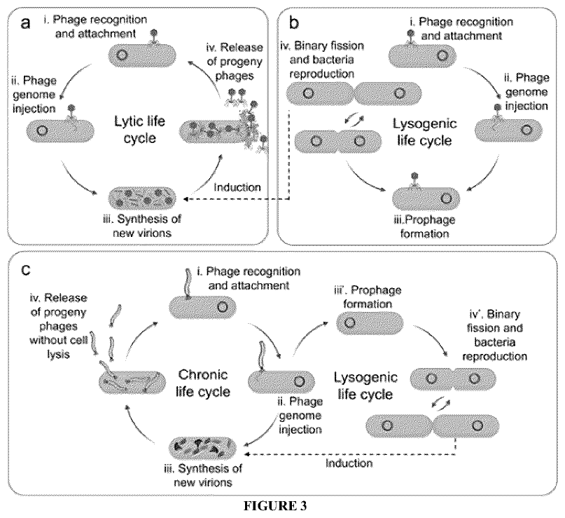

It is important to note here that phages may have at least three different cycles, namely lytic, lysogenic, and chronic (FIG. 3). Through the lytic cycle (FIG. 3a) a phage can lyse a bacterial cell in as short as ~20 minutes, leading to the release of the bacterial intracellular content in addition to tens or hundreds of progeny phages.²⁴ Regulatory agencies have historically only approved strictly lytic phages for human therapeutic use and environmental biocontrol because of outstanding concerns regarding horizontal gene transfer through the lysogenic cycle (FIG. 3b,c).²⁵ As our aim was to screen phage libraries for therapeutic potential, we focused our work on the detection of phages capable of bacterial lysis.

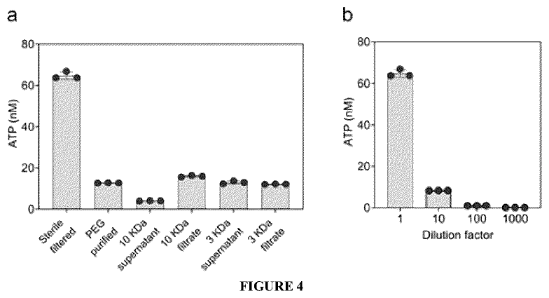

Increasing signal-to-noise ratio and end-point detection of phage-mediated bacteria lysis. The next step towards realizing the high throughput, one-pot detection of phage-mediated bacteria lysis was to filter out the background signal. Phage stocks are obtained through the infection and lysis of bacterial cells leading to the release of progeny phages along with other intracellular components, including ATP. Therefore, we hypothesized that phage suspensions must be treated to reduce residual ATP molecules which can otherwise give rise to a strong background signal. The goal was to diminish the background bioluminescent signal at time zero, i.e., at the point of phage addition to the bacterial culture, which would in turn decrease the assay time and allow a signal to be discernable as soon as phage-mediated lysis of bacterial cells occurs. FIG. 4a shows the background bioluminescence signal in phage suspensions at different stages of purification (including sterile filtration with 0.2 μm filters, PEG purification, supernatant, and ultra-filtration with 10 KDa and 3 KDa filters) and after diluting phages in fresh culture media. After PEG purification, the ATP concentration decreased significantly; however, ultrafiltration with 3 KDa filters failed to reduce ATP levels. As shown in FIG. 4b, simply diluting concentrated phage suspensions in cell media was found to be equally effective for reducing background ATP signal as the labour-intensive PEG purification technique Based on these results, we used the dilution method to reduce background bioluminescence signal and for preparing phage libraries. While the dilution method reduced the residual ATP signal, it did not completely eliminate the background bioluminescence signal. Thus, the background signal was deducted from the data for better visualization of the signal resulting from phage-induced ATP release.

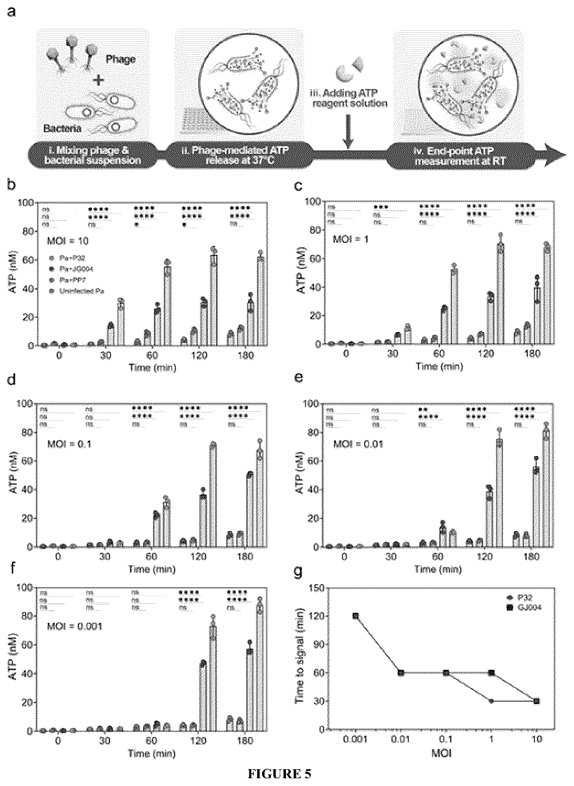

For end point detection of phage lysis, each phage was mixed with a culture of host bacteria at physiological temperature. Aliquots were collected periodically and added to ATP assay reagents in a stagewise manner at room temperature, before measuring the bioluminescence signal (FIG. 5a). Exponentially growing bacteria were infected with phage at MOIs of 10, 1, 0.1, 0.01, and 0.001. FIG. 5 b-f show ATP release as a result of phage-mediated lysis, measured over a 3-hr period. At MOI~10, bioluminescence signal was detected within 30 mins after the addition of phages P32 or JG004 to the bacterial culture (FIG. 5b). At lower MOIs, bioluminescence signal became detectable at later time points. The phage with the weakest lytic activity, PP7, showed significant bioluminescence signal only at MOI=10 and after 60 to 120 mins, while at lower MOIs, the signal was not significantly different from the uninfected Pa control, demonstrating the ability of the assay to differentiate strong and week lytic activity. Another trend to note is that for P32 and JG004 phages at MOIs of 0.01 and 0.001 (FIG. 5e, f), the signal intensity at the end of 3 hrs incubation period was visibly higher than the same time point at MOI=10. This can be explained by the lower number of phages at the beginning of infection, which provides enough times for bacterial cells to grow and increase their population, FIG. 5g clearly shows that shortest time to detect a positive signal is with the highest MOI.

Another noteworthy trend is that even at very low phage to bacteria ratio of 1:1000 (MOI=0.001), signal intensity from phage infected samples is different from uninfected samples after two hours of incubations (FIG. 5f). This likely shows the ability of the ATP detection biochemistry to discriminate between phage-mediated lysis and bacterial autolysis at very low MOIs. It is important to keep in mind that the concentration of phages curated in libraries is quantified in terms of plaque forming units, which reflects the number of infective particles capable of infecting the known host bacteria used to propagate the library phages at the stage of making the library. When screening for therapeutic phages against an unknown strain of bacteria (e.g., a multidrug resistant clinical or environmental isolate), the number of infective particles against an uncharacterized strain may be orders of magnitude lower than the original host strain, a concept known as efficiency of plaquing.²⁶ Therefore, demonstrating susceptibility profiling at low MOI (low efficacy of plaquing) is an additional advantage.

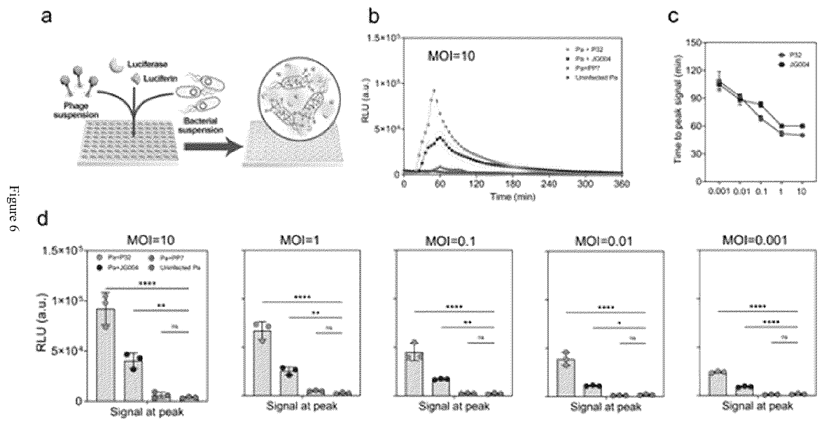

One-pot biochemistry for detection of phage lytic activity. End-point detection of phage-mediated bacteria lysis involves periodic sampling of bacterial cultures, mixing with ATP reagents at room temperature, before measuring the bioluminescence signal. This technique is time-consuming and laborious, making it incompatible for high throughput screening applications. A one-pot format in which assay reagents are mixed with bacterial cultures at the start of phage infection cycle (FIG. 6a) would be a more desirable and practical approach when designing a high throughput screening technology. FIG. 6b shows the bioluminescence kinetic curve for cultures infected with phage P32 or phage JG003 at MOI=10 in a one-pot format. Notably, a detectable signal appears ~30 mins post infection and peaks at 60 mins. Thereafter, the signal decays quickly until it becomes undetectable ~4 hours post-infection. Signal detected from cultures infected with phage PP7, known to have weak lytic activity, was indistinguishable from uninfected cultures. The major shortcoming of the one-pot biochemistry of ATP detection, however, was that the bioluminescence signal produced under these conditions started to decay after 1 hour (FIG. 6b). The signal decay is likely the result of thermal inactivation of luciferin and luciferase at 37° C.

Despite the temperature inactivation of luciferase, our data show that the one-pot format not only discriminates between uninfected and infected cultures but can also differentiate between strong (P32), moderate (JG004), and weak (PP7) lytic activity of different phages even at initial lower phage titers (lower MOIs) (FIG. 6d). This capability to resolve lytic activity makes our method as efficient as the gold standard plaque assay, while offering the obvious advantage of being amenable to high throughput format. A re-examination of FIG. 1d showed that the plaque morphology for each phage, which shows the combined effect of phage burst size and latent period and is thus a good indicator (although not the only indicator) for phage biocontrol efficacy, agrees with the trends observed in FIGS. 6b and d. Latent periods and burst size of phages are among the characteristics used to evaluate phage virulence,²⁷ but they may not be indicative of therapeutic potential in vivo as various conditions will affect the efficacy of phage therapy as mentioned by other researchers.²⁸ Phage P32 is reported to have a 20-minute latent period and a burst size of 210 progeny phages on P. aeruginosa.¹⁵ JG004 is reported to have a 31-minute latent period and a burst size of 13 progeny phages on P. aeruginosa.¹⁶ Latent period and burst size numbers may vary depending on host bacterial strain, stage of bacterial growth, and nutrient source; however, the general trend should hold. FIG. 6b shows 25-30 mins are required from the time of phage addition to the time to detect bioluminescence signal, which matches closely with the latent period of phages P32 and JG004.20 As seen in FIG. 6c, the time to peak signal decreases as concentration of phage increases (higher MOIs) as expected. The fact that phage P32 generated a stronger peak signal as compared to phage JG004 showed that more bacterial cells were lysed by P32 compared to JG004 (FIG. 6d). The peak signal correlates with the number of bacterial cells lysed. Having a large burst size can also affect the peak signal by having more progeny phages released to the surrounding medium, that can in turn infect and lyse more bacterial cells. Taken altogether, these findings confirm the feasibility of the one-pot chemistry for the detection of phage-mediated cell lysis and thus showing promise to form the basis of a high throughput susceptibility profiling technology.

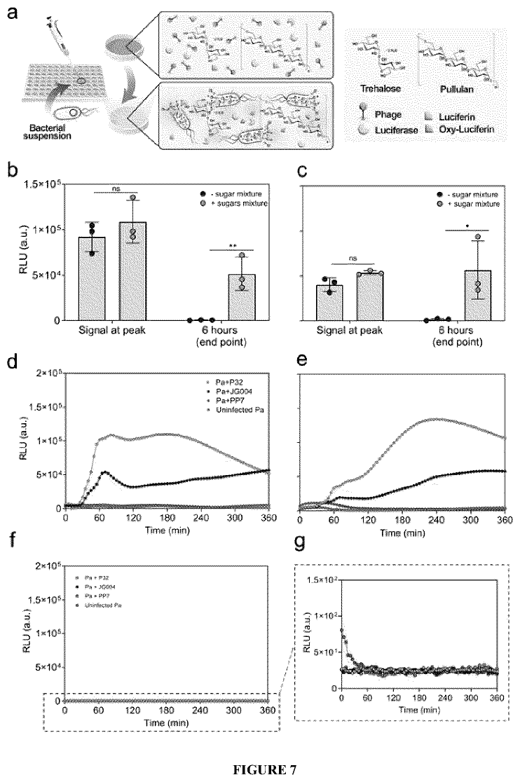

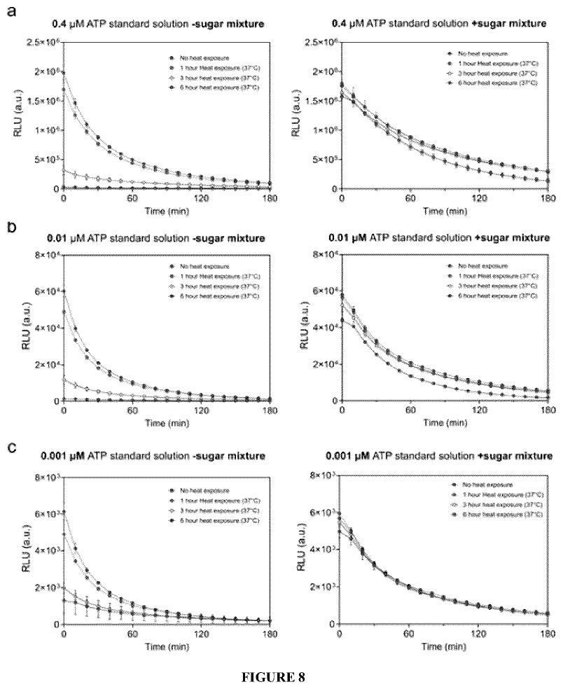

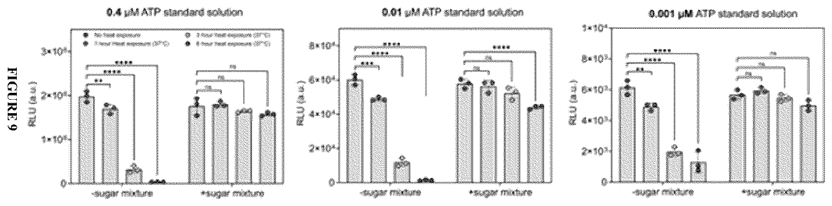

Optimizing detection of phage-mediated lysis in a stabilizing sugar polymer matrix. The next step towards a reliable susceptibility assay was to address the rapid enzyme deactivation at physiological temperatures. To address this challenge, we selected pullulan, based on previously reported thermo-protective effects towards biomolecules,^{29,30} and trehalose based on reported desiccation protection towards viruses.^{31,32} Pullulan and trehalose were added to a mixture of phage suspension and lyophilized ATP detection reagents and homogenized before the addition of bacteria (FIG. 7a). As shown in FIG. 7d, a discernable signal appeared 25-30 mins after all assay components were mixed with the bacterial suspension. The time to signal detection in the presence of the sugar mixture was comparable to the sugar-free mixture (FIG. 6b,c). This confirmed that the addition of sugar polymers did not interfere with the function of assay reagents or with phage infectivity. Moreover, the addition of sugars (pullulan-trehalose) did not significantly affect the peak bioluminescence signal, whereas the signal at 6 hrs post infection in cultures infected with either phages P32 (FIG. 7b) or JG004 (FIG. 7c) was ~99% and 95% higher, respectively, than the negative control. In summary, addition of pullulan-trehalose to the mixture stabilized the assay signal and significantly reduced signal decay. To further assess how closely the RLU signal follows the phage-mediated cell-lysis, we designed an experiment with ATP standard solutions. ATP reaction solutions were prepared in the absence or presence of the sugar polymer mixture. ATP standard solutions with three concentrations (0.4, 0.01, 0.001 μM) were prepared in water. The RLU values correlated with 0.4 μM ATP standard solution was chosen at least 10 times larger than the amount of ATP released in the solution for the phage-mediated ATP detection in our one-pot assays. 0.01 and 0.001 μM ATP standard solutions were tested to see if at lower ATP values we are still able to detect consistent values in real-time phage mediated RLU signal monitoring at 37° C. for weak lysis activity. The activity of the ATP reagent solution with and without sugar polymers were also tested in four conditions including no previous exposure at 37° C. (tested right after preparation at room temperature), one, three and 6 hours after incubation at 37° C. ATP standard solutions were added to the above-mentioned treated and untreated ATP reactions in the presence and absence of the sugar mixture, and the signal was measured at time zero, followed by continuous measurements for up to 3 hours. The results confirmed that sugar polymer was able to stabilize the ATP bioluminescence assay at 37° C., and the loss of signal was not significant after exposure at 37° C. in all three ATP standard concentrations. On the other hand, in the absence of sugars, the ATP reagent solution led to a significant decrease in RLU signal after incubation at 37° C. (FIG. 8). In addition, the kinetic RLU signal monitoring showed that the rate of signal loss was also higher when no sugar was present in the ATP reagent solution (FIG. 8). Specifically, the loss of signal after 6 hours incubation at 37° C. resulted in an approximately 90% signal loss (FIG. 9). This confirms that ATP bioluminescence assay in the presence of sugar polymers has been able to detect the ATP release as a result phage-mediated bacterial cell lysis.

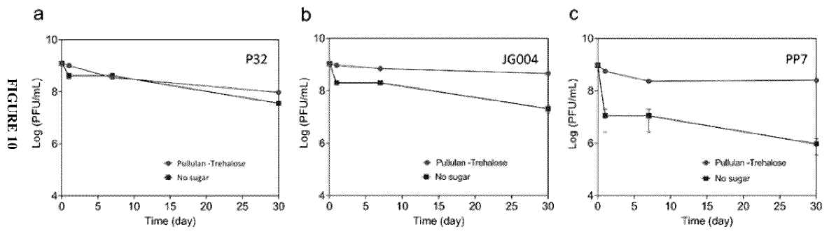

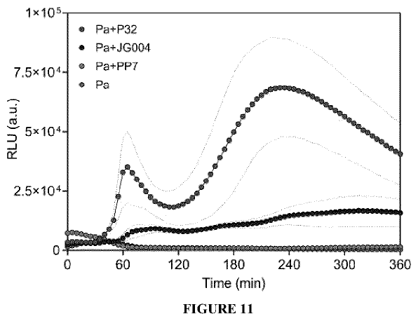

We further demonstrated that the sugar mixture can be dried and cast into a tablet format, encasing all the assay components into a sugar matrix. As shown in FIG. 10, titer loss for phage encased in the sugar polymer matrix was less than one log for all three phages after a 30-day storage under ambient condition. In the absence of the sugar polymer matrix, phages showed weak desiccation tolerance, with PP7 being the least stable one with a 3-log reduction after 30 days. We then evaluated the stability of all assay reagents in a dehydrated tablet form. ATP assay components were first mixed with pullulan-trehalose and then phage suspensions in a well plate and dried under nitrogen airflow. Following a week-long storage under vacuum, dried tablets were reconstituted. As shown in FIG. 7e, the rate of bioluminescence signal from the reconstituted tablet assay was slower compared to the liquid assays. Although the signal intensity was lower compared to the original liquid assay, the RLU signal of the phage containing tablets compared to control uninfected Pa was visibly higher showing a successful detection of the phage-mediated bacterial cell lysis. The signal intensity depends on the dissolution rate of the tablets in water, which in turn controls the release of phages and ATP reagents into the mixture. We believe that the slower increase of the bioluminescence signal from the reconstituted tablet is a small trade-off for the portability, improved storage, and ease of use afforded by the solid tablet format (FIG. 7d). The all-inclusive tablets also preserved their stability after four weeks of storage at room temperature under vacuum (FIG. 11).

Lastly, it should be noted that the drying of the ATP reagents and phages in the absence of sugar polymers led to a complete loss of signal after the one-week storage period, as shown in FIG. 7f, g. This clearly shows the importance of sugar polymers in preserving phages and ATP assay components, which is particularly advantageous for ease of transportation and point-of-use accessibility, including disaster scenarios as well as underdeveloped and remote regions.

Screening different bacterial species against phage libraries using sugar-based ATP bioluminescence assay. We have selected four different bacterial species, namely P. aeruginosa, S. enterica, E. coli, and S. aureus with two strains per species to assess the feasibility of the ATP bioluminescence assay in identifying target phages against bacterial strain of interest. The information regarding all the strains is included in Table 1.

TABLE 1
List of bacterial species and strains

| Bacteria | | Identifier | Source | Note |
|---|---|---|---|---|
| P. aeruginosa | PAO1 | DSM 22644 | DSMZ | NA |
| | C0072² | NA | Urine* | Antibiotic resistance profile: |
| | | | | ampicillin, amoxicillin clavulanic |
| | | | | acid, cefazolin, cefalotin, |
| | | | | cefixime, nitrofuratoin, |
| | | | | tetracycline, trimethoprim |
| | | | | sulfamethoxazole, cefoxitin, |
| | | | | ceftriaxone |
| | C0335² | NA | Arm* | ampicillin, amoxicillin clavulanic |
| | | | | acid, cefazolin, cefalotin, |
| | | | | cefixime, nitrofuratoin, |
| | | | | tetracycline, trimethoprim |
| | | | | sulfamethoxazole, cefoxitin, |
| | | | | ceftriaxone |
| Salmonella | S. Newport | HER1019 | Sewage** | NA |
| | C487 | | | |
| | S. Senftenberg | HER1397 | Human | NA |
| | S-219/89 | | blood** | |
| E. coli | O157:H7 | HER1262 | Feces** | NA |
| | B1190-1 | | | |
| | LF82 | NA | Ileal | NA |
| | | | Mucosa of | |
| | | | Crohn's | |
| | | | disease | |
| | | | patient¹, | |
| | | | provided | |
| | | | by Dr. | |
| | | | Brian | |
| | | | Coombes | |
| S. aureus | S. aureus 68 | HER 1049 | NA** | NA |
| | S. aureus 44A | HER 1101 | NA** | NA |

*Clinical isolates from the in-house library at the Michael DeGroote Institute of Infectious Disease Research.
**From the Felix D'Herelle Reference Center for Bacterial Viruses

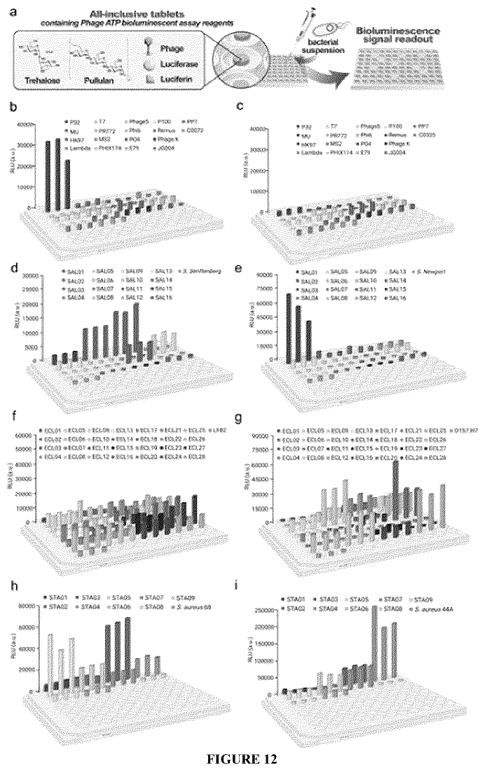

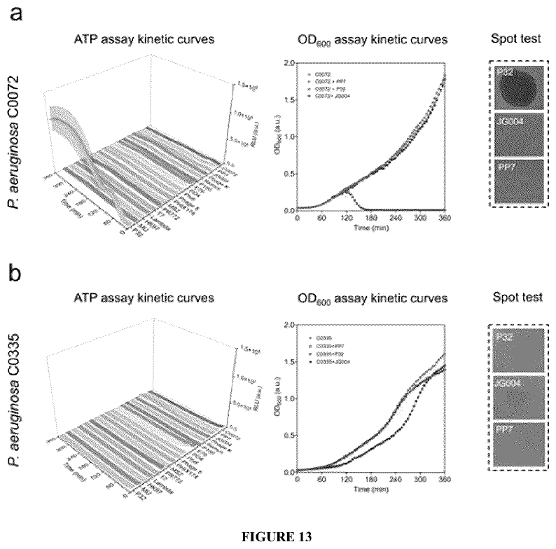

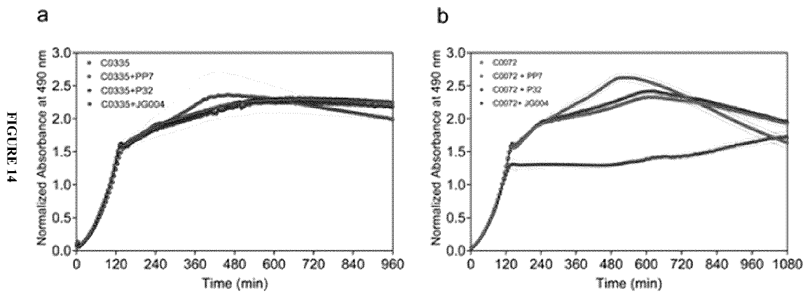

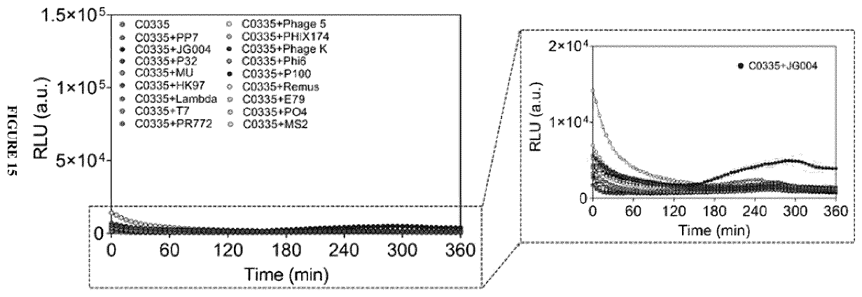

We randomly selected two multidrug resistant clinical isolates of P. aeruginosa from an in-house library of isolates from Hamilton Health Sciences. The antibiotic resistance profile for these strains is shown in Table 1.³³ The two strains were given the designations C0072 and C0335 and were isolated from patients with urinary tract infection and arm wound infection respectively. We conducted phage susceptibility screening against our in-house phage library containing seventeen phages, with mixed host strains to control for false positive signals, using one week-old, all-inclusive tablets stored under ambient conditions. FIG. 12a shows the general workflow for these assays. The kinetic RLU signal monitoring results of the tablet-based, one-pot assay shows a significant, but slow rise in bioluminescence signal after 30 minutes for P. aeruginosa C0072 infected with P32 (FIG. 13a). As snapshot of the signals from the microtiter plate is illustrated in FIG. 12b after 90 min, showing strong signals form the identified phages. As shown in FIG. 13b, for the clinical isolate C0335, the bioluminescence assay did not show any rise in signal with any of the phages in the library (also demonstrated in FIG. 12c). However, a faint signal was observed for one of the phages (JG004) with a delay. Phage susceptibility was confirmed with optical density assay, and a spot test (representative images shown in FIG. 13a, b) as well as XTT metabolic activity monitoring assay (FIG. 14). The clinical isolate C0072 showed an obvious clearance with P32 (FIG. 13) but clinical isolate C0335 isolate did not show susceptibility to any of the phages in the library, although a very faint clearing was observed with JG004 (FIG. 13, spot test), which may correspond to the faint signal after 180 min (FIG. 15). The identified phage for C0335 is clearly very weak and thus not recommended for phage therapy/biocontrol applications. We calculated the efficiency of plaquing of the identified phage on C0072 to be 0.77, confirming a high infection efficiency.³⁴ These data demonstrate that tablets, stored under ambient conditions, could identify phages capable of infecting clinical bacterial isolates. These data illustrate that the all-inclusive solid tablet format is capable of rapid screening MDR isolates against phage libraries with high signal to noise ratio and reliability comparable to gold standard culture techniques, as well as metabolic assays.

To further challenge our platform technology, we screened two different strains of E. coli (O157:H7 and LF82) against a library of 28 E. coli phages, two strains of S. enterica (serovar Newport and serovar Senftenberg) against a library of 16 Salmonella phages, and two strains of S. aureus against a library of nine S. aureus phages from the Felix D′Herelle Reference Center for Bacterial Viruses, in the presence of sugar polymers. A full list of coded phages present in these two libraries can be found in Table 2, 3 and 4. The received phages had titers of 108-1010PFU/mL and were used without dilution to closely mimic a real life scenario where our technology is proposed to be used, when the initial titers in the library are not known for the bacteria of interest.

TABLE 2
List of S. enterica phages

| Phages | Codes |
|---|---|
| HER535 S16 | SAL01 |
| HER 40 01 | SAL02 |
| STL4 | SAL03 |
| HER20 16 | SAL04 |
| HER95 X | SAL05 |
| HER19 7-11 | SAL06 |
| HER428 1 Heidelberg | SAL07 |
| HER415 SasL6 | SAL08 |
| HER39 Vill | SAL09 |
| HER431 4 Heidelberg | SAL10 |
| HER397 SasL4 | SAL11 |
| ER429 2 Heidelberg | SAL12 |
| HER437 10 Heidelberg | SAL13 |
| HER340 Sab2 | SAL14 |
| HER161 P22 | SAL15 |
| HER45 Jersey | SAL16 |

TABLE 3
List of E. coli phages

| Phage name | Codes |
|---|---|
| HER139 HK243 | ECL01 |
| HER144 Lambda | ECL02 |
| HER263 9 | ECL03 |
| HER261 7 | ECL04 |
| HER26 T3 | ECL05 |
| HER24 T1 | ECL06 |
| HER158 RB69 | ECL07 |
| HER77 N4 | ECL08 |
| HER268 14 | ECL09 |
| HER270 16 | ECL10 |
| HER536 Alpha3 | ECL11 |
| HER267 13 | ECL12 |
| HER266 12 | ECL13 |
| HER265 11 | ECL14 |
| HER256 2 | ECL15 |
| HER255 1 | ECL16 |
| HER586 BRET | ECL17 |
| HER468 T1uV | ECL18 |
| HER315 Ω8 | ECL19 |
| HER337 O103 | ECL20 |
| HER42 pt1 | ECL21 |
| HER27 T4 | ECL22 |
| HER382 HK97 | ECL23 |
| HER364 SS4 | ECL24 |
| HER258 4 | ECL25 |
| HER257 3 | ECL26 |
| HER259 | ECL27 |
| HER262 | ECL28 |

TABLE 4
List of S. aureus phages

| Phage name | Codes |
|---|---|
| HER225 3A | STA01 |
| HER226 77 | STA02 |
| HER49 P68 | STA03 |
| HER101 44AHJD | STA04 |
| HER528 Remus | STA05 |
| HER474 K | STA06 |
| HER475 812 | STA07 |
| HER239 187 | STA08 |
| HER238 71 | STA09 |

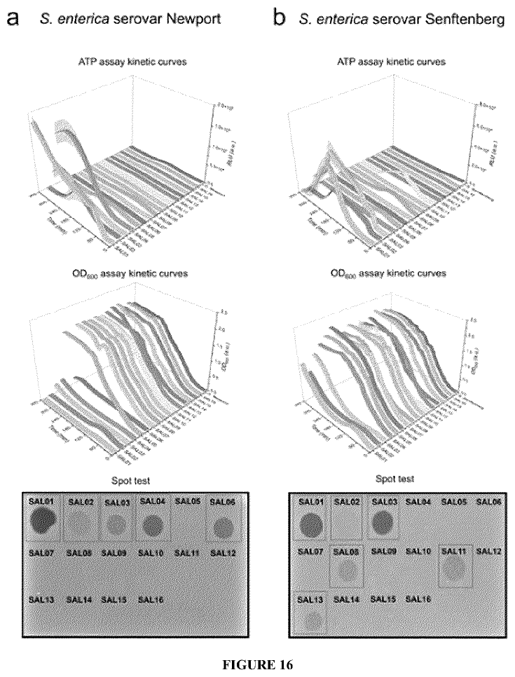

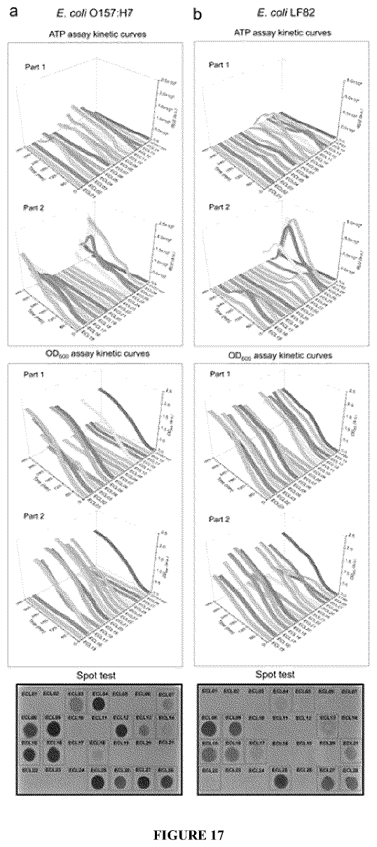

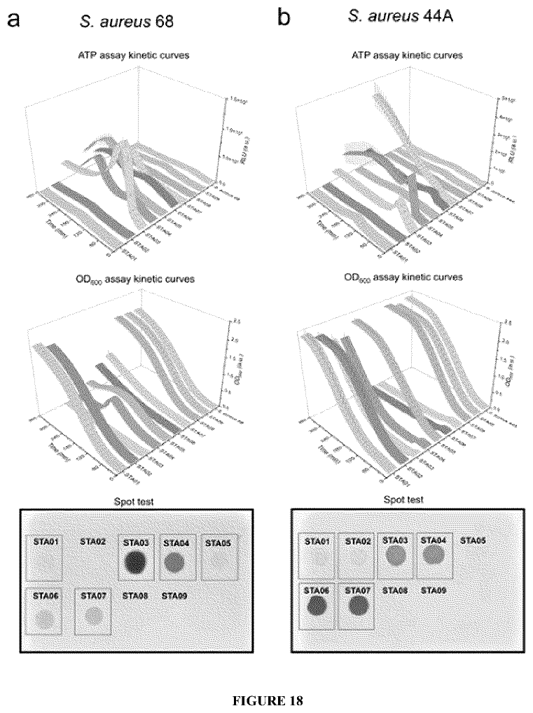

FIGS. 12d and e shows a snapshot of the signals from the salmonella phage library after 90 min, with signals form identified targets clearly higher than the background signal. Examining the kinetic bioluminescence curves (FIG. 16) show that the signal to noise ratio is very high at 90 min, clearly marking the phages that lead to lysis of the bacterial cell and burst release of ATP. The same trend can be observed for the E. coli phage library, where the kinetic bioluminescence curves (FIG. 17) show that phages causing lysis of the bacterial cell and burst release of ATP are identified with a high signal to noise ratio within 100 min (3D snapshot presented in FIGS. 12f and g). In the S. aureus phage library, five phages were identified withing 170 min against the S. aureus 68 strain (FIG. 5h). However, a wide range of response times observed when screening this strain against our phage library as evident in the kinetic one-pot ATP curves (FIG. 18a). Among the five target phages against S. aureus 68 strain, STA03 and STA06 showed ATP signal within 30 min and STA04 within 60 min. On the other hand, STA05, and STA07, both with very weak spot test signal, showed bioluminescence signal at later time within 130 min and 170 min, respectively. For S. aureus 44A, all the target phages were identified within 100 min (FIG. 5i). The kinetic one-pot ATP curves for S. aureus 44A is shown in FIG. 18b.

In addition, we benchmarked our method against gold standard culture methods, namely spot tests on semi solid media (end point detection) and kill curves (monitoring culture optical density at 600 nm, OD600). These data are presented in FIGS. 16, 17 and 18 and show strong agreement with culture methods, verifying the reliability of our platform. Examining the data summarized for comparison with bioluminescence assay in Tables 5, 6 and 7 further reveals the utility of ATP detection as a direct measure of bacterial lysis, as opposed to indirect measures such as kill curves, specifically that certain phages that showed a strong signal with kill curves, showed no ATP signal, and no or very faint spot test clearing. What gives the ATP detection method an edge over other components is that it relieves a major bias shared by optical density and metabolic activity monitoring, a notable example being detection of slowed growth not accompanied by bacterial lysis (which happens for chronic phage or in some cases of lysogeny) versus bacterial cell lysis and destruction. The burst of ATP detected in our method can only happen if the bacterial cell is compromised and thus the detection of phage-mediated ATP release directly detects cell lysis, while other methods measure infectivity indirectly by monitoring the phenotypic features of bacterial cultures. Moreover, ATP release shows a real time measurement of bacterial cell lysis which can provide information on phage dormant period on a particular bacterial strain.

TABLE 5
Comparative rating of the ATP detection method with
culture techniques for Salmonella phages

| | | | |
|---|---|---|---|
| Targets identified | ATP detection | OD kill curve | Spot test |

Salmonella enterica Serovar Newport

| | | | |
|---|---|---|---|
| SAL01 | +++ | +++ | +++ |
| SAL02 | ++ | +++ | + |
| SAL03 | ++ | +++ | + |
| SAL04 | +++ | +++ | ++ |
| SAL06 | No | +++ | ++ |

Salmonella enterica Serovar Senftenberg

| | | | |
|---|---|---|---|
| SAL01 | +++ | +++ | +++ |
| SAL02 | ++ | +++ | − |
| SAL03 | +++ | +++ | +++ |
| SAL08 | + | + | + |
| SAL11 | + | + | + |
| SAL13 | − | + | + * |

+++: strong signal (ATP assay: the highest and continuous RLU signal detected upon strain infection within the phage library, OD₆₀₀: the lowest turbidity detected upon strain infection within the phage library in comparison with negative control (uninfected strain), Spot test: clear plaque on the strain's bacterial lawn)
++: medium signal (ATP assay: a medium RLU signal detected upon strain infection in comparison with the highest RLU value within the phage library, OD₆₀₀: the medium reduction in turbidity detected upon strain infection in comparison with negative control, Spot test: turbid plaque on the strain's bacterial lawn)
+: weak signal (ATP assay: a weak RLU signal value detected upon strain infection in comparison with the highest and medium RLU value within the phage library, OD₆₀₀: a weak reduction in turbidity detected upon strain infection in comparison with negative control, Spot test: faint plaque on the strain bacterial lawn
* very faint footprint

TABLE 6
Comparative rating of the ATP detection method
with culture techniques for E. coli phages

| | | | |
|---|---|---|---|
| Targets identified | ATP detection | OD kill curve | Spot test |

E. coli O157: H7

| | | | |
|---|---|---|---|
| ECL03 | + | + | + |
| ECL04 | + | +++ | +++ |
| ECL07 | + | +++ | + |
| ECL08 | + | +++ | +++ |
| ECL09 | + | +++ | +++ |
| ECL10 | ++ | +++ | NA * |
| ECL12 | + | +++ | +++ |
| ECL13 | + | +++ | + |
| ECL14 | + | +++ | + |
| ECL15 | +++ | +++ | +++ |
| ECL16 | +++ | +++ | +++ |
| ECL18 | ++ | ++ | + |
| ECL25 | + | +++ | +++ |
| ECL26 | +++ | +++ | ++ |
| ECL27 | + | +++ | +++ |
| ECL28 | +++ | +++ | +++ |

E. coli LF82

| | | | |
|---|---|---|---|
| ECL04 | + | + | + |
| ECL07 | + | No | No |
| ECL08 | + | + | +++ |
| ECL09 | ++ | ++ | +++ |
| ECL12 | + | No | No |
| ECL13 | No | No | + ** |
| ECL15 | + | ++ | ++ |
| ECL16 | + | ++ | ++ |
| ECL17 | + | + | + |
| ECL21 | No | +++ | + *** |
| ECL22 | No | +++ | + *** |
| ECL23 | + | No | No |
| ECL24 | + | No | No |
| ECL25 | +++ | +++ | +++ |
| ECL26 | + | No | No |
| ECL27 | +++ | +++ | +++ |
| ECL28 | +++ | +++ | +++ |

+++: strong signal (ATP assay: the highest and continuous RLU signal detected upon strain infection within the phage library, OD₆₀₀: the lowest turbidity detected upon strain infection within the phage library in comparison with negative control (uninfected strain), Spot test: clear plaque on the strain's bacterial lawn)
++: medium signal (ATP assay: a medium RLU signal detected upon strain infection in comparison with the highest RLU value within the phage library, OD₆₀₀: the medium reduction in turbidity detected upon strain infection in comparison with negative control, Spot test: turbid plaque on the strain's bacterial lawn)
+: weak signal (ATP assay: a weak RLU signal value detected upon strain infection in comparison with the highest and medium RLU value within the phage library, OD₆₀₀: a weak reduction in turbidity detected upon strain infection in comparison with negative control, Spot test: faint plaque on the strain bacterial lawn
* The kill curve and ATP detection methods exhibit relatively strong signals, leading to the conclusion that the spot test in this case may be biased.
** very faint footprint
*** no plaques detected upon serial dilution.

TABLE 7
Comparative rating of the ATP detection method
with culture techniques for S. aureus phages

| | | | |
|---|---|---|---|
| Targets identified | ATP detection | OD kill curve | Spot test |

S. aureus 68

| | | | |
|---|---|---|---|
| STA01 | No | No | +* |
| STA03 | +++ | +++ | +++ |
| STA04 | ++ | ++ | ++ |
| STA05 | ++ | ++ | +* |
| STA06 | ++ | ++ | +* |
| STA07 | ++ | ++ | +* |

S. aureus 44A

| | | | |
|---|---|---|---|
| STA01 | No | + | +* |
| STA02 | No | + | +* |
| STA03 | ++ | ++ | ++ |
| STA04 | +++ | ++ | ++ |
| STA06 | ++ | +++ | ++ |
| STA07 | +++ | +++ | ++ |

+++: strong signal (ATP assay: the highest and continuous RLU signal detected upon strain infection within the phage library, OD₆₀₀: the lowest turbidity detected upon strain infection within the phage library in comparison with negative control (uninfected strain), Spot test: clear plaque on the strain's bacterial lawn)
++: medium signal (ATP assay: a medium RLU signal detected upon strain infection in comparison with the highest RLU value within the phage library, OD₆₀₀: the medium reduction in turbidity detected upon strain infection in comparison with negative control, Spot test: turbid plaque on the strain's bacterial lawn)
+: weak signal (ATP assay: a weak RLU signal value detected upon strain infection in comparison with the highest and medium RLU value within the phage library, OD₆₀₀: a weak reduction in turbidity detected upon strain infection in comparison with negative control, Spot test: faint plaque on the strain bacterial lawn
*very faint footprint

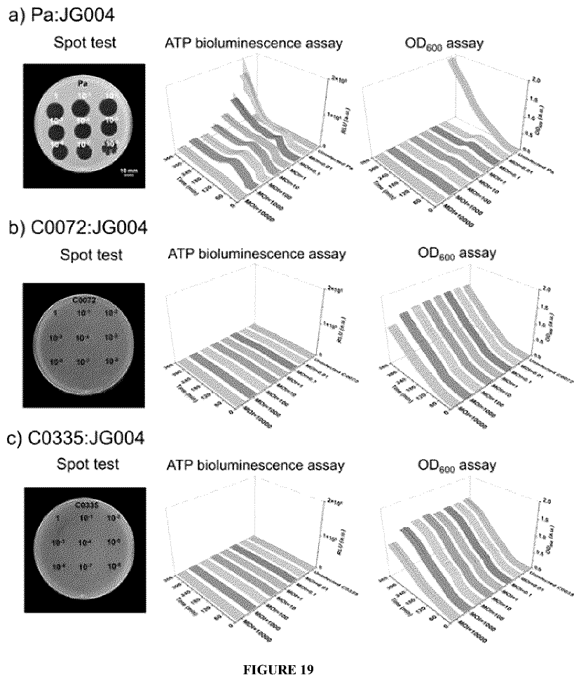

At high MOIs, some phages can bind to the bacterial strain and lead to bacterial death through abortive infection without releasing progeny phages, a phenomenon known as “lysis from without”. To investigate whether cell death without progeny phage production can be a source of bias in the phage screening process, we expanded the range of MOI in our investigation. Lysis from without is reported to happen at higher MOIs (>100 phages per bacterium),^{35,36} and thus we infected three P. aeruginosa strains with high titers of phage JG004 at a wide range of MOIs (~10,000, 1000, 100, 1, 0.1, and 0.01). As shown in FIG. 19, only the Pa strain was sensitive to phage JG004, and the other three strains did not show any clearance on bacterial lawns (spot test) or a decrease in turbidity in standard kill curves, or any increase in RLU signal in ATP bioluminescence assay at any of the MOI's tested (FIG. 19b, c, d). As shown in FIG. 19a, in the kill curves, turbidity increased at the beginning of the assay and peak turbidity decreased with increase in MOI, which could be partially attributed to lower number of uninfected bacterial cells at high phage concentration and partially to lysis from without. For the phage sensitive Pa strain, the kill curves at different MOIs barely exhibit any difference, with the growth being effectively hindered at every MOI and the curve being visually indiscernible at MOI's≥1.

The signal from ATP bioluminescence assays, however, showed very interesting trends, highlighting the power of direct monitoring of phage-mediated bacterial lysis (as opposed to indirect monitoring with turbidity assays). The Pa strain infected with an extremely high MOI of ~10,000, showed a significantly lower ATP bioluminescence signal compared to lower MOIs. The peak signal increased by lowering the MOI in the range of 10,000 to 10. However, the peak signal started to diminish by lowering the MOI further, while showing larger signal at the endpoint of the assay. It is noteworthy that lysis from without usually happens earlier than normal time of lysis (<5 min), but it can also happen at normal lysis times,³⁵ which is in line with our observation. The time to signal that we obtained for MOIs ≥10 was around 30 mins and increased to 60, 70 and 90 mins for MOIs 1, 0.1, and 0.01, respectively.

The observed trend in bioluminescence signal (FIG. 19) can be explained based on the dynamic of phage-bacteria interactions. When phage concentration is extremely high, some abortive phage infection may happen due to lysis from without. It has been reported in the literature that at high MOIs, lysis from without can lead to reduced phage counts and cell death.³⁵ This may be the reason behind the observed decrease in bioluminescence signal at MOI>10. At MOI<1 the signal at the endpoint was larger as the phage population was smaller and some uninfected bacteria (theoretically 37% of the population)³⁷ could continue growing, which would lead to a higher signal at the end for MOI=0.01.

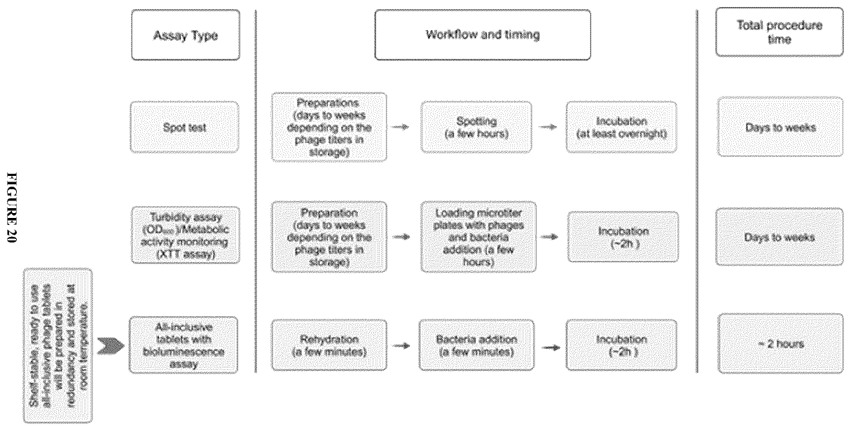

The proposed new method is an all-in-one solution that is amenable to high throughput implementation, automation, and stable to store and shipping with small footprint and without the need for a cold chain. We envision phage libraries stabilized and stored in 384-well microtiter plates along with the biochemistry of cell lysis detection, in redundancy. When needed, these microtiter plates will be shipped to the point-of-use, where the only task needed will be to rehydrate and add the target bacteria; both steps are automation-friendly and take seconds to minutes (not accounting for bacterial growth of the initial bacterial culture, which is the same for all other methods). This is all in contrast to conventional culture-based liquid assays (kill curves), other liquid assays (e.g. XTT assay), and semi-solid assays (spot test) for which the phage biobank is seldom replicated and shipped to point-of need, for susceptibility screening. Because some phages may be unstable during shipping, hosts (pathogenic) strains may also need to be shipped. At the point of screening, phages will have to be retrieved from storage (in a freezer, fridge or freeze-dried) one-by-one, either added to liquid bacterial culture (tube, microtiter plate) along with the host bacteria and any other reagents that may be needed, such as redox sensitive dyes in case of an XTT assay, or divalent cations in any phage susceptibility assay of choice. In case of a spot test, the solid medium will have to be prepared in advance, which will add to the laborious preparation process. Even an ATP assay would be cumbersome and lengthy without the proposed tablets, since all reagents will have to be freshly added in a stage-wise fashion, and the assay will have to be run at room temperature, which would significantly slow down the growth of pathogenic bacteria and phage-mediated lysis, while increasing assay time. The phage stocks stored in conventional format may lose titer from one to multiple logs; so, it is standard practice to check titer prior to screening, which will add to the required time. FIG. 20 illustrates the overall screening time needed for our proposed method in comparison with the spot test, turbidity measurements, for a modest size library of ~100 phages and one person dedicated to the task.

### DISCUSSION

Personalized phage therapy is becoming an alternative strategy for many patients suffering from infections resistant to all known antibiotics. However, access to phage therapy is challenged by the lack of a universal phage library. There have been efforts in curating phage libraries around the world, but due to the remarkable phage diversity, and the numerous challenges associated with maintaining such biobanks, the more realistic forecast is that we may always need to screen multiple phages located in decentralized libraries find suitable phage(s) against a resistant infection. This is challenged also by the lack of rapid screening methods, which are laborious, time-consuming, with a slow response time. Here, we addressed both challenges and presented a path towards an improved routine practice of personalized phage therapy worldwide.

We re-imagined phage libraries not in the form of liquid lysates in a fridge, or even frozen stocks in a deep freezer, but as solid tablets packed in microtiter plates, stored on a shelf, ready for rapid high throughput screening with a plate reader when needed, and ready to ship with a moment's notice. Each tablet contains a phage stock along with enzymes and ions needed to detect the burst of ATP release during phage-mediated bacteria lysis, all stabilized in a sugar polymer matrix that protects the enzymes against degradation at physiological temperatures. The matrix also offers desiccation protection to phages and enzymes, making the tablets shelf stable and easy to store and to ship. In addition, the solid, all-inclusive tablet format eliminates the need for stage-wise addition of reagents, enabling high throughput screening.

The proposed approach has obvious advantages over the culture techniques for phage susceptibility profiling, namely a faster response time and compatibility with high throughput implementation, as well as environmental stability, eliminating the need for a cold chain or special packaging. It also offers a point-of-use implementation with minimal infrastructure and training, which will be particularly impactful in remote regions. Our technology goes beyond binary susceptibility profiling and can also semi-quantitatively determined the phage ability for biocontrol. With personalized phage therapy being increasingly practiced for treating antibiotic resistant infections, these proposed all-inclusive phage-containing tablets will speed up the screening process to identify phages that target the pathogenic bacterial strain of interest.

### Materials

Pullulan (PI20 food grade, 200 kDa) from Hayashibara Co, Ltd., Okayama, Japan was kindly provided by Dr. Carlos Filipe, Department of Chemical Engineering, McMaster University. D-(+)-Trehalose dehydrate, and XTT tetrazolium sodium salt, menadione, and acetone were purchased from Sigma Aldrich. Phosphate-buffered saline (PBS) tablets were purchased from VWR (Mississauga, ON, CA). Luria Broth (LB) and Tryptic Soy Broth (TSB) were purchased from Fisher Scientific (ON, CA). ATP bioluminescence Assay Kit CLS II was purchased from Millipore Sigma (Sigma-Aldrich, Oakville, ON). vB_Pae-Tbilisi32 (P32) and JG004 phages, and PAO1 (Pa) strain were purchased from DSMZ (Germany), and PP7 phage and E. coli 157:H7 bacterial strain from Université Laval (QC, Canada). LF82 was generously provided by Dr. Brian Coombes (Department of Biochemistry & Biomedical Science, McMaster University). Two clinical P. aeruginosa strains including C0072 and C0335 were obtained from IIDR database at McMaster University (Please refer to Table 1 for additional information). Salmonella strains along with 16 salmonella phages, 28 E. coli phages, and two S. aureus strains along with 9 S. aureus phages were obtained from the Felix d′Herelle Reference Center for Bacterial Viruses at Universite Laval (https://www.phage.ulaval.ca/en/home/). The details of bacterial species and phages can be found in the Tables 1, 2, 3 and 4.

### Methods

Bacterial culture and phage propagation. All frozen bacterial stocks were stored at −80° C. in 25% v/v glycerol. Overnight bacterial cultures were prepared by inoculating 3 mL of bacterial media with glycerol stock. P. aeruginosa and E. coli strains were cultured in LB media, while S. enterica and S. aureus strains were grown in TSB media. The inoculated media was incubated at 37° C. and 180 rpm for 16-18 hours to promote bacterial growth. Overnight cultures were subsequently diluted 1:100 in 50 mL of fresh bacterial cell media and incubated for 2-3 hours to allow bacterial cells to reach the mid-exponential growth phase. 10 μL of stock phage solution was introduced to bacterial cells at mid-exponential phase and the cultures were incubated for a further 6 hours to allow for phage lysis of bacterial cells. The lysate was centrifuged for 20 min at 7000 rcf, and the supernatant was sterilized using a 0.2-μm-pore-sized filters and stored at 4° C. For E. coli and Salmonella phages propagation, overnight bacterial cultures were grown in 10 mL of TSB from glycerol stocks at 37° C. with agitation. Bacterial overnight culture was diluted 1:100 in 10 mL of TSB and a scratch of phages from glycerol stocks was added. The mixture was incubated at 37° C. with agitation until lysis (around 6 hours). Lysates were filtered through 0.45 μm filters.

Phage concentration (number of plaque forming units per milliliter, PFU/mL) was determined using the overlay technique.³⁸ Phage stock was serially diluted in LB or TSB media and plated on bacterial lawns to enumerate the number of infectious virions present in the stock.

Efficiency of plating (EOP). Efficiency of plating measures the titer of phage infecting a non-host bacterial strain. EOP assay was conducted with P32 (propagated on host strain, Pa) for P. aeruginosa C0072 strain. Phage overlay assay was conducted in triplicates to obtain the PFUs on the test strain (C0072) and the host strain (Pa). EOP was calculated using the following formula.³⁴

$$EOP : \frac{Average PFU on the test strain}{Average PFU on the host bacterium ( Pa )} \\ Infection efficiency : \\ EOP \geq 0.5 ( high production efficiency ) \\ 0.1 \leq EOP < 0.5 ( moderate production efficiency ) \\ 0.001 < EOP < 0.1 ( Low production efficiency ) \\ EOP \leq 0.001 ( inefficient )$$

Phage purification. The aqueous two-phase method was used to purify filter-sterilized phage suspension.³⁹ Briefly, sterile 20 (w/v) % Poly(ethylene glycol) containing 2.5 M NaCl solution was added to the phage stock suspension at a ratio of 1:6 v/v followed by overnight incubation at 4° C. Phages in suspension was pelleted by centrifugation at 5000 rcf for 45 minutes and resuspended in 10 mL of RO Millipore water and subjected to mild agitation at 4° C. for at least 2 hours. PEG purified samples went through further purification using Amicon Ultra centrifugal filters (Millipore Sigma, Ultra-15, MWCO 10 KDa, and 3 KDa). ATP background signal was measured at different stages of purification and compared with starting phage suspension to monitor the change in background noise. For all liquid phage infection assays, the initial titer of each phage was determined using the double agar layer method by three independent experiments.

XTT metabolic assay. XTT assay is commonly used to measure metabolic activity of cells using a tetrazolium salt which is a formazan compound in the presence of metabolically active cells followed by a detectable change of color. Assays were conducted in clear, flat-bottom, 96-well plates with total volume of 100 μL in each well. 12 μL of phage suspensions (~109-105 PFU/mL) were mixed with 38 μL bacteria at OD₆₀₀ ~0.1 and added to wells at the final multiplicity of infection (MOI) of 10, 1, 0.1, 0.01, and 0.001. XTT solution was prepared in LB media to contain 0.2 mg/mL of XTT and 0.1 mM menadione. 50 μL of the XTT solution was added to all wells, followed by the addition of 50 μL of LB media as negative control, 12 μL of media with 38 μL of bacterial suspension as positive control, and 12 μL of phage with 38 μL bacterial suspension as phage-infected sample. Absorbance was measured at 490 nm wavelength every 5 minutes for at least16 hours using Synergy Neo2 BioTek plate reader set at 37° C. (n=3). The absorbance value from wells containing XTT solution with LB media was subtracted from bacteria-containing wells.

Optical density assay (OD600). The overnight culture of bacterial strain was added to fresh LB or TSB media at 1:100 v/v ratio and incubated until the sub-culture reached OD₆₀₀ ~0.1. Assays were conducted in transparent flat bottom 96-well plate with total assay volume of 200 μL per well. 45 μL of Phage solutions (~109 to 105 PFU/mL) with 155 μL of bacteria at OD₆₀₀ ~0.1 (~3×10⁷ CFU/mL) were added to specific wells at the final multiplicity of infection (MOI) of 10, 1, 0.1, 0.01, and 0.001. Control wells contained 155 μL bacteria and 45 μL bacterial media. Absorbance was measured at 600 nm wavelength using Synergy Neo2 BioTek plate reader (n=3). Data was collected every 5 minutes for at least 16 hours at 37° C. OD₆₀₀ numbers was calculated by subtracting the absorbance of media, divided by the pathlength correction obtained from BioTek plate reader.

Transmission electron microscopy (TEM). Phages with titers of 109 PFU/mL were absorbed onto plasma-cleaned carbon-coated copper grids and negatively stained with 1% uranyl acetate. Stained grids were dried at room temperature and imaged using Talos L120C transmission electron microscope at the Canadian Centre for Electron Microscopy (CCEM), McMaster University (ON, CA).

ATP Bioluminescence assay. ATP assay reagent solution was by reconstituting the lyophilized ATP reagent solution using bioluminescence Assay Kit CLS II (Sigma-Aldrich, Oakville, ON) based on manufacturer's instruction. For conducting ATP assay in pullulan-trehalose sugar solution, lyophilized ATP reagents of the ATP bioluminescence Assay Kit CLS II were reconstituted with the sterile sugar solution containing 10 wt % pullulan and 0.5 M trehalose, at the same liquid volume as manufacturer's instruction.

Assays were conducted in white flat bottom 96-well plate with total assay volume of 100 μL per well. Bioluminescence signal was detected from wells containing 50 μL of ATP reagent solution mixed with 50 μL of the sample (phage infected or uninfected bacterial suspension). 12 μL of phage suspensions with ~10⁹ to 10⁵ PFU/mL and 38 μL of bacteria at OD₆₀₀ ~0.1 (~3×10⁷ CFU/mL) was used for obtaining MOIs of 10, 1, 0.1, 0.01. 0.001.

For end-point ATP bioluminescent assay, the ATP reagent solutions were added at the end point to samples and the signal was measured at room temperature. The RLU signal was measured at various times points (0, 30, 60, 120, and 180 minutes) using Synergy Neo2 BioTek plate reader (n=3). The ATP standard curves were prepared per manufacturer's instruction to calculate the ATP amounts.

For one-pot ATP bioluminescent assay, 50 μL of ATP reagent solution was added to specified wells, followed by 12 μL of phage suspensions at different concentrations (10⁹-10⁵ PFU/mL) and 38 μL bacterial sub-cultures at OD₆₀₀=0.1 respectively, to obtain specific MOIs (10, 1, 0.1, 0.01, 0.001) within a final volume of 100 μL per assay (n=3). The plates containing ATP assay reagent solution, phage-infected, and uninfected bacterial cultures were incubated at 37° C. inside a Synergy Neo2 BioTek plate reader and the RLU signal was recorded every 5-10 minutes in stationary state without shaking to prevent damaging the sensitive ATP reagents. Wells with uninfected bacterial suspensions, and wells containing phage only were used as controls. The RLU values from phage-only wells were subtracted from the data when further dilution of the samples would lead to significant titer loss.

The integration time was kept constant at 5 seconds for all the endpoint and one-pot ATP bioluminescence measurements.

Stabilization in sugar polymer matrices. Sugar solutions containing 10 wt % pullulan and 0.5 M trehalose was dissolved in milli-Q water and autoclaved to sterilize.

ATP bioluminescence assay reagent stability. To assess the stability of ATP bioluminescence assay reagent in sugar solution, the ATP assay reagents were prepared in 10 wt % pullulan and 0.5 M trehalose, and the activity was tested with ATP standard solution at 37° C. Three concentrations of ATP standard solution (0.4, 0.01, 0.001 μM) were prepared in water. To evaluate the ATP reagent solution's activity, both with and without sugar polymers, four conditions were examined: immediate testing after preparation at room temperature (no prior exposure at 37° C.), testing one hour after incubation at 37° C., testing three hours after incubation at 37° C., and testing 6 hours after incubation at 37° C. Subsequently, ATP standard solutions were introduced into the treated and untreated ATP reaction solution, in the presence and absence of the sugar mixture. The signal was initially measured at time zero, followed by continuous measurements every 10 minutes for up to 3 hours to comprehensively compare the rate of signal decay in the absence and presence of sugars (n=3).

Phage stability. Phage suspensions were diluted in sugar solution to achieve a titer of 109 PFU/mL, and 100 μL of the phage suspension were added to 24-well plates and air-dried. Stability of phages within dried pullulan-trehalose matrix was tested. Phage titer was calculated 1-, 7-, and 30-days after storage at room temperature using the plaque overlay assay method (n=3).

All-inclusive dried tablet-based ATP assay. To stabilize the ATP reagents and the phage particles in a dried format, the plate containing phages (15 μL of 10⁹ PFU/mL) and ATP reagent solution (50 μL) in sugars solution was added to specified wells. The plate was dried under nitrogen flow in a glove bag for at least 4 hours, followed by storage under vacuum at −0.08 MPa. The Stability of dried tablets containing ATP reagent solution and different phages in 96 well-plate was assessed after one- and 4-weeks storage at room temperature. For conducting the assay, the dried tablet containing the ATP reagents and phage particles embedded in sugar polymer matrix were rehydrated using 50 μL of sterile milli-Q water, followed by addition of 50 μL bacteria sub-culture at OD₆₀₀=0.1 (~3×10⁷ CFU/mL), and reading the signal every 5 minutes at 37° C. As a control, a plate containing phages and ATP reagent solution was prepared without addition of sugar polymers, and dried/stored under same conditions. The plate was rehydrated and tested following the same process used for assessing the phage-ATP reagent solution encased in sugar matrices.

Phage library screening using all-inclusive ATP bioluminescence assay in sugar polymer matrices. To screen the clinical isolates of P. aeruginosa, including C0072 and C0335, the in-house phage library including 16 phages from different species (Pseudomonas phages including P32, JG004, PP7, E79, PO4, and Phi6; Staphylococcus aureus phages including phage K and Remus, E. coli phages including T7, MS2, HK97, PR772, Lambda, PHIX174, phage 5 and MU, and Listeria P100 phage) was used. 10 μL of each phage was mixed with 50 μL of ATP reagent solution containing 10 wt % pullulan and 0.5 M trehalose in wells of a white flat-bottom 96 well plate. The control well for uninfected bacterial control contained 50 μL of sugar-based ATP reagent solution and 10 μL of LB media. The assay reagents along phages were desiccated under nitrogen flow in a glove bag for at least 4 hours, followed by storage under vacuum at −0.08 MPa for one week. On the day of assay, tablets were rehydrated with 50 μL sterile milli-q water, followed by addition of 40 μL bacteria sub-cultures of each clinical strain (including C0072 and C0335) grown in LB media at OD₆₀₀=0.1 (~3×10⁷ CFU/mL), and reading the signal every 5 minutes for up to 6 hours at 37° C. All the phages had an approximate titer in the range of 10⁸-10⁹ PFU/ml. (n=3)

To screen the library of Salmonella phages, 10 μL of each phage from the library (Table 2) with initial titers above ~10⁸ PFU/mL and 10 μL of TSB media for negative control wells (uninfected bacterial culture) were added to 50 μL of ATP reagent solution containing 10 wt % pullulan and 0.5 M trehalose in wells of a white flat-bottom 96 well plate. Afterwards, 40 μL of Salmonella bacterial sub-culture of (S. Newport C487 and S. Senftenberg S-219/89) grown in TSB media to OD₆₀₀=0.1 were added to each well. The plates were incubated at 37° C. inside a Synergy Neo2 BioTek plate reader and the RLU signal was recorded every 6 minutes in stationary state without shaking to prevent damaging the sensitive ATP reagents. (n=3).

To screen the E. coli phage library against LF82 and O157:H7, we followed the same screening steps as those employed for the salmonella phage library. 40 μL of sub-cultures of E. coli LF82 and O157:H7 grown in LB media at OD600=0.1 were added to wells containing 50 μL of ATP reagent solution containing 10 wt % pullulan and 0.5 M trehalose, and 10 μL of each phage in wells of a white flat-bottom 96 well plate. The list of E. coli phages can be found in Table 3. The RLU signals were read every 10 minutes at 37° C. using a Synergy Neo2 BioTek plate reader. (n=3).

Similar to other strains, to screen the S. aureus phage library, 40 μL of sub-cultures of S. aureus 68 HER 1049 and S. aureus 44A HER 1101 strains grown in TSB media at OD₆₀₀=0.1 were added to wells containing 50 μL of ATP reagent solution containing 10 wt % pullulan and 0.5 M trehalose, and 10 μL of each phage in wells of a white flat-bottom 96 well plate. The list of S. aureus phages can be found in Table 4. The RLU signals were read every 10 minutes at 37° C. using a Synergy Neo2 BioTek plate reader. (n=3)

Modeling conditions for lysis from without. Three P. aeruginosa strains namely PAO1 (Pa), C0072 and C0335 (Table 1) were infected at MOIs 10000, 1000, 100, 10, 1, 0.1 and 0.01. For spot test, JG004 phage with initial concentration of ~5×10¹¹ PFU/mL was serial diluted (10-fold) from a dilution factor of 1 to 10⁻⁸ and 10 μL of each dilution was spotted on a bacterial lawn of P. aeruginosa strains.

For ATP bioluminescence assay, 40 μL bacterial sub-cultures at OD₆₀₀=0.1 (~10⁷ CFU/mL) was added to wells of a 96-well plate containing 50 μL of ATP reagent solution in 10 wt % pullulan and 0.5 M trehalose, mixed with 10 μL of phage suspensions at different concentrations (5×10¹¹-5×10⁵ PFU/mL), to obtain specific MOIs (10000, 1000, 100, 10, 1, 0.1 and 0.01) within a final volume of 100 μL per assay (n=3). The plate was incubated at 37° C. inside a Synergy Neo2 BioTek plate reader and the RLU signal was recorded every 5 mins. Optical density (OD600) assay was conducted at the same MOIs by adding 80 μL of bacterial subcultures at OD₆₀₀=0.1 (~10⁷ CFU/mL) to 20 μL of phage at different concentrations (5×10¹¹-5×10⁵ PFU/mL) and 100 μL of LB media to a final volume of 200 μL to obtain MOIs of 10000, 1000, 100, 10, 1, 0.1 and 0.01 while keeping the final phage/bacteria concentration same as the one-pot ATP bioluminescence assay (n=3). The absorbance at 600 nm was recorded at 37° C. every 5 minutes using inside a Synergy Neo2 BioTek plate reader. Wells with uninfected bacterial suspensions were used as controls in both assays.

### REFERENCES

1. Watts, J. E. M., Schreier, H. J., Lanska, L. & Hale, M. S. The rising tide of antimicrobial resistance in aquaculture: Sources, sinks and solutions. Mar. Drugs 15, 1-16 (2017).
2. Fish, R. et al. Bacteriophage treatment of intransigent Diabetic toe ulcers: A case series. J. Wound Care 25, S27-S33 (2016).
3. Ganeshan & Hosseinidoust. Phage Therapy with a focus on the Human Microbiota. Antibiotics 8, 131 (2019).
4. Hsu, B. B. et al. Dynamic Modulation of the Gut Microbiota and Metabolome by Bacteriophages in a Mouse Model. Cell Host Microbe 25, 803-814.e5 (2019).
5. Mu, A. et al. Assessment of the microbiome during bacteriophage therapy in combination with systemic antibiotics to treat a case of staphylococcal device infection. Microbiome 9, 92 (2021).
6. Schooley, R. T. et al. Development and use of personalized bacteriophage-based therapeutic cocktails to treat a patient with a disseminated resistant Acinetobacter baumannii infection. Antimicrob. Agents Chemother. 61, (2017).
7. Ramirez-Sanchez, C. et al. Successful treatment of Staphylococcus aureus prosthetic joint infection with bacteriophage therapy. Viruses 13, 1-10 (2021).
8. Ács, N., Gambino, M. & Brøndsted, L. Bacteriophage Enumeration and Detection Methods. Front. Microbiol. 11, (2020).
9. Letarov, A. V. & Kulikov, E. E. Determination of the bacteriophage host range: Culture-Based approach. Methods Mol. Biol. 1693, 75-84 (2018).
10. Xie, Y., Wahab, L. & Gill, J. J. Development and validation of a microtiter plate-based assay for determination of bacteriophage host range and virulence. Viruses 10, (2018).
11. Henry, M. et al. Development of a high throughput assay for indirectly measuring phage growth using the OmniLog T M system. Bacteriophage 2, 159-167 (2012).
12. McCallin, S., Sacher, J. C., Zheng, J. & Chan, B. K. Current state of compassionate phage therapy. Viruses 11, 1-14 (2019).
13. Merril, C. R., Scholl, D. & Adhya, S. L. The prospect for bacteriophage therapy in Western medicine. Nat. Rev. Drug Discov. 2, 489-497 (2003).
14. Koksharov, M. I. & Ugarova, N. N. Thermostabilization of firefly luciferase by in vivo directed evolution. Protein Eng. Des. Sel. 24, 835-844 (2011).
15. Karumidze, N. et al. Characterization of lytic Pseudomonas aeruginosa bacteriophages via biological properties and genomic sequences. Appl. Microbiol. Biotechnol. 94, 1609-1617 (2012).
16. Garbe, J., Bunk, B., Rohde, M. & Schobert, M. Sequencing and Characterization of Pseudomonas aeruginosa phage JG004. BMC Microbiol. 11, 1-12 (2011).
17. Kim, E. S. et al. cDNA-Derived RNA Phage Assembly Reveals Critical Residues in the Maturation Protein of the Pseudomonas aeruginosa Leviphage PP7. J. Virol. 95, (2021).
18. Hyman, P. & Abedon, S. T. Bacteriophage Host Range and Bacterial Resistance. in Advances in Applied Microbiology 70, 217-248 (Elsevier Inc., 2010).
19. Burrowes, B. H. Bacteriophages, Biology, Technology, Therapy. (Springer International Publishing, 2021).
20. Hyman, P. & Abedon, S. T. Practical methods for determining phage growth parameters. Methods Mol. Biol. 501, 175-202 (2009).
21. Abedon, S. T. Detection of Bacteriophages: Phage Plaques. in Bacteriophages 507-538 (Springer International Publishing, 2021).
22. Abedon, S. T. & Katsaounis, T. I. Basic phage mathematics. Methods Mol. Biol. 1681, 3-30 (2018).
23. Weiss, M. et al. In vivo replication of T4 and T7 bacteriophages in germ-free mice colonized with Escherichia coli. Virology 393, 16-23 (2009).
24. Abedon, S. T., Herschler, T. D. & Stopar, D. Bacteriophage Latent-Period Evolution as a Response to Resource Availability. Appl. Environ. Microbiol. 67, 4233-4241 (2001).
25. Hyman, P. Phages for phage therapy: Isolation, characterization, and host range breadth. Pharmaceuticals 12, (2019).
26. Kutter, E. Chapter 14-Phage host range and efficiency of plating. Bacteriophages Methods Protoc. Vol. 1 Isol. Charact. Interact. 501, 141-149 (2009).
27. Gill, J. & Hyman, P. Phage Choice, Isolation, and Preparation for Phage Therapy. Curr. Pharm. Biotechnol. 11, 2-14 (2010).
28. Dąbrowska, K. & Abedon, S. T. Pharmacologically Aware Phage Therapy: Pharmacodynamic and Pharmacokinetic Obstacles to Phage Antibacterial Action in Animal and Human Bodies. Microbiol. Mol. Biol. Rev. 83, (2019).
29. Jahanshahi-Anbuhi, S. et al. Simple and ultrastable all-inclusive pullulan tablets for challenging bioassays. Chem. Sci. 7, 2342-2346 (2016).
30. Jahanshahi-Anbuhi, S. et al. Pullulan encapsulation of labile biomolecules to give stable bioassay tablets. Angew. Chemie-Int. Ed. 53, 6155-6158 (2014).
31. Leung, V. et al. Long-Term Preservation of Bacteriophage Antimicrobials Using Sugar Glasses. ACS Biomater. Sci. Eng. 4, 3802-3808 (2018).
32. Leung, V., Groves, L., Szewczyk, A., Hosseinidoust, Z. & Filipe, C. D. M. Long-Term Antimicrobial Activity of Phage-Sugar Glasses is Closely Tied to the Processing Conditions. ACS Omega 3, 18295-18303 (2018).
33. Alcock et al. 2023. CARD 2023: Expanded Curation, Support for Machine Learning, and Resistome Prediction at the Comprehensive Antibiotic Resistance Database. Nucleic Acids Research, 51, D690-D699.
34. Mirzaei, M. K. & Nilsson, A. S. Isolation of phages for phage therapy: A comparison of spot tests and efficiency of plating analyses for determination of host range and efficacy. PLOS One 10, 1-13 (2015).
35. Abedon, S. T. Lysis from without. Bacteriophage 1, 46-49 (2011).
36. Dennehy, J. J. & Abedon, S. T. Phage Infection and Lysis. in Bacteriophages 1-43 (Springer, 2020). doi: 10.1007/978-3-319-40598-8_53-1
37. Abedon, S. T. & Katsaounis, T. I. Basic Phage Mathematics. in Bacteriophages. Methods in Molecular Biology 3-30 (Springer, 2018). doi: 10.1007/978-1-4939-7343-9_1
38. Green, M. R. & Sambrook, J. Molecular cloning: A Laboratory Manual (Fourth edition). Cold Spring Harbor Laboratory Press 1, (John Inglis, 2012).
39. Sambrook, J. & Russell, D. W. Molecular cloning: A laboratory manual. (Cold Spring Harbour Laboratory Press, 2001).

1. A combination comprising (1) a composition comprising a sugar-based matrix, wherein the sugar-based matrix encapsulates a phage and (2) a reagent for detecting a bacterial cell condition.

2. The combination of claim 1, wherein the bacterial cell condition is cell growth, cell metabolic activity, cell death, cell membrane integrity, cell lysis, or any combination thereof.

3. The combination of claim 1, wherein the bacterial cell condition is bacterial cell lysis.

4. The combination of claim 1, wherein the sugar-based matrix comprises a sugar with a T_{g} of greater than about 40 degrees C.

5. The combination of claim 1, wherein the sugar-based matrix comprises a sugar without reducing groups.

6. The combination of claim 1, wherein the sugar-based matrix comprises an oligosaccharide and a disaccharide sugar.

7. The combination of claim 1, wherein the sugar-based matrix comprises a sugar that is a desiccant.

8. The combination of claim 7, wherein the sugar comprises trehalose.

9. The combination of claim 1, wherein the sugar-based matrix comprises pullulan, maltodextrin, dextran, inulin, alginate, cellulose, trehalose, sucrose, lactose, maltose, or any combination thereof.

10. The combination of claim 9, wherein the sugar-based matrix comprises pullulan and trehalose.

11. The combination of claim 1, wherein the reagent detects ATP and/or XTT.

12. The combination of claim 11, wherein the reagent comprises XTT tetrazolium salt (2,3-Bis-(2-Methoxy-4-Nitro-5-Sulfophenyl)-2H-Tetrazolium-5-Carboxanilide) and menadione.

13. The combination of claim 1, wherein the composition is stable at room temperature for at least four weeks under vacuum and/or wherein the composition demonstrates susceptibility profiling at low MOI (low efficacy of plaquing).

14. (canceled)

15. The combination of claim 1, wherein the bacterial cell condition is detected through a colour change, fluorescence, and/or bioluminescence.

16. The combination of claim 15, wherein the generated bioluminescence signal is increased by up to about 90% as compared to a composition without the sugar-based matrix.

17. The combination of claim 1, wherein the composition provides for phage susceptibility screening in less than about 120 minutes.

18. The combination of claim 1, wherein the sugar-based matrix enhances desiccation tolerance of the phage and/or the reagent.

19. The combination of claim 1, wherein the combination is in a single composition and wherein the composition is in solid or gel form, or is printed or coated on a surface.

20. (canceled)

21. A high throughput phage screening platform comprising:
a) a plurality of the combinations of claim 1, wherein each combination comprises a different phage; or
b) a portable library of individual shelf-stable, ready to use phages, in solid tablets, wherein the tablets optionally contain reagents for detecting a bacterial cell condition.

22-23. (canceled)

24. A method for detecting phage-mediated bacterial cell lysis, the method comprising applying a bacterial culture to the combination of claim 1.

25-34. (canceled)
